(19) United States
(12) Patent Application Publication
(10) Pub. No.: US 2026/0183447 A1
(43) Pub. Date: Jul. 2, 2026

(54) METHOD FOR FORMING MEDICAL DEVICES FROM A METAL PLATE

(71) Applicant: MiRus LLC, Marietta, GA (US)

(72) Inventor: Noah Roth, Marietta, GA (US)

(21) Appl. No.: 19/380,457

(22) Filed: Nov. 5, 2025

Related U.S. Application Data
(60) Provisional application No. 63/739,236, filed on Dec. 27, 2024.

(51) Int. Cl. A61L 27/04; B22F 1/17; B22F 3/10

(52) U.S. Cl. CPC A61L 27/047; B22F 1/17; B22F 3/1017; A61L 2420/02

(57) ABSTRACT

A method and process for manufacturing medical devices from a flat planar metal plate.

### REFERENCED APPLICATIONS

The present disclosure claims priority on U.S. Provisional Application Ser. No. 63/739,236 filed Dec. 27, 2024, which is incorporated herein by reference.

### FIELD OF DISCLOSURE

The disclosure relates generally to medical devices, more particularly to a method for forming a medical device from a metal plate and then optionally further processing the medical device by bending one or more portions of the medical device.

### BACKGROUND

When manufacturing medical devices such as, but not limited to bone plates, it is difficult to efficiently manufacture such medical devices in a timely and cost effective manner, while also maintaining the stringent dimension parameters of such medical devices. The present disclosure is directed to a method for manufacturing medical devices in a timely and cost effective manner, while also maintaining the stringent dimension parameters of such medical devices.

### SUMMARY OF THE DISCLOSURE

The present disclosure is direct to a method for manufacturing medical devices in a timely and cost effective manner, while also maintaining the stringent dimension parameters of such medical devices.

In one non-limiting aspect of the present disclosure, there is a method and process for manufacturing medical devices (e.g., bone plates and other orthopedic medical devices) from flat metal plates. In one non-limiting embodiment, the method and process for manufacturing medical devices includes the steps of a) providing a flat planar metal plate (e.g., plate has 0-2° curvature and all values and ranges therebetween), and wherein the cross-sectional area of the metal plate and the thickness of the metal plate are non-limiting, and wherein the thickness of the metal plate is constant over 80-100% (and all values and ranges therebetween) of the cross-sectional area of the metal plate, b) cutting and/or machining the metal plate using one or more processes (e.g., water jet cutting, laser cutting, EDM cutting, CNC machining, electrochemical etching, grinding, etc.) to form one or more medical devices from the metal plate, c) optionally separating the one or more medical devices from the metal plate (e.g., cutting the one or more medical devices from the metal plate that was not used to form the one or more medical devices, shearing the one or more medical devices from the metal plate that was not used to form the one or more medical devices, grinding off one or more medical devices from the metal plate that was not used to form the one or more medical devices, etc.), d) optionally smoothing the edges (e.g., smoothing the outer perimeter edges of the medical device, smoothing the edges of one or more openings that are spaced from the perimeter edge of the medical device, etc.) by use of one or more processes (e.g., polishing, grinding, etc.), e) optionally grinding down and smoothing the one or more regions of the medical device that needed to be cut or separated by some other method when the medical device was separated from the metal plate, and f) bending (e.g., bending by use of a hydraulic press, bending by use of mating dies, etc.) the one or more medical devices to form a final shape of the one or more medical devices.

In one non-limiting aspect of the present disclosure, there is a method and process for manufacturing medical devices (e.g., bone plates and other orthopedic medical devices) from flat metal plates. In one non-limiting embodiment, the method and process for manufacturing medical devices includes the steps of a) providing a flat planar metal plate (e.g., plate has 0-2° curvature and all values and ranges therebetween), and wherein the cross-sectional area of the metal plate and the thickness of the metal plate are non-limiting, and wherein the thickness of the metal plate is constant over 80-100% (and all values and ranges therebetween) of the cross-sectional area of the metal plate, b) cutting and/or machining the metal plate using one or more processes (e.g., water jet cutting, laser cutting, EDM cutting, CNC machining, drilling, gun drilling, electrochemical etching, shearing, grinding, etc.) to form one or more medical devices from the metal plate, c) optionally separating the one or more medical devices from the metal plate (e.g., cutting the one or more medical devices from the metal plate that was not used to form the one or more medical devices, shearing the one or more medical devices from the metal plate that was not used to form the one or more medical devices, grinding off one or more medical devices from the metal plate that was not used to form the one or more medical devices, etc.), d) optionally smoothing the edges (e.g., smoothing the outer perimeter edges of the medical device, smoothing the edges of one or more openings that are spaced from the perimeter edge of the medical device, etc.) by use of one or more processes (e.g., polishing, grinding, etc.), e) optionally grinding down and smoothing the one or more regions of the medical device that needed to be cut or separated by some other method when the medical device was separated from the metal plate, f) bending (e.g., bending by use of a hydraulic press, bending by use of mating dies, etc.) the one or more medical devices to form a final shape of the one or more medical devices, g) optionally further cutting (e.g., water jet cutting, laser cutting, EDM cutting, CNC machining, drilling, gun drilling, electrochemical etching, shearing, grinding, etc.) the medical device to form the final form and shape of the medical device, and wherein this optional cutting process can occur prior to and/or after the step of bending, and h) optionally applying a coating to a portion or all of the outer surface of the medical device prior to and/or after the bending of the medical device into its final shape.

In one non-limiting aspect of the present disclosure, there is provided a flat planar metal plate (e.g., plate has 0-2° curvature and all values and ranges therebetween). The top and/or bottom surface of the metal plate can optionally be polished to have a smooth top and/or bottom surface. In one non-limiting embodiment, the shape of the metal plate is square or rectangular; however, other shapes can be used. In another and/or alternative non-limiting embodiment, the thickness of the metal plate can be constant over 80-100% (and all values and ranges therebetween) of the cross-sectional area of the metal plate. In another and/or alternative non-limiting embodiment, the thickness of the metal plate is non-limiting. In one non-limiting configuration, the thickness of the metal plate is 0.01 mm to 10 mm (and all values and ranges therebetween). Prior to the metal plate being cut, the plate can be optionally covered and/or sealed in a material (e.g., plastic, etc.) to inhibit or prevent oxidation of the surface of the plate. Prior to the further processing of the plate (e.g., cutting, bending, coating, etc.) the material used for sealing, when used, is typically removed.

In accordance with another and/or alternative non-limiting aspect of the present disclosure, the metal plate can be formed of a variety of materials. In one non-limiting embodiment, the metal plate is partially (e.g. 1-99.999 wt. % and all values and ranges therebetween) or fully formed of a metal alloy that includes a) stainless-steel, b) CoCr alloy, c) TiAlV alloy, d) aluminum alloy, e) nickel alloy, f) titanium alloy, g) tungsten alloy, h) molybdenum alloy, i) copper alloy, j) beryllium-copper alloy, k) titanium-nickel alloy, l) refractory metal alloy, m) metal alloy (e.g., stainless-steel, CoCr alloy, TiAlV alloy, aluminum alloy, nickel alloy, titanium alloy, tungsten alloy, molybdenum alloy, copper alloy, beryllium-copper alloy, titanium-nickel alloy, refractory metal alloy, etc.) that is modified to further include at least 5 atomic weight percent (awt. %) or atomic percent (awt. %) rhenium (e.g., 5-99 awt. % rhenium and all values and ranges therebetween), or n) metal alloy (e.g., stainless-steel, CoCr alloy, TiAlV alloy, aluminum alloy, nickel alloy, titanium alloy, tungsten alloy, molybdenum alloy, copper alloy, beryllium-copper alloy, titanium-nickel alloy, refractory metal alloy, etc.) that is modified to further include at least 5 atomic weight percent (awt. %) or atomic percent (awt. %) hafnium (e.g., 5-99 awt. % hafnium and all values and ranges therebetween). As used herein, atomic weight percent (awt. %) or atomic percentage (awt %) or atomic percent (awt. %) are used interchangeably. As defined herein, the weight percentage (wt. %) of an element is the weight of that element measured in the sample divided by the weight of all elements in the sample multiplied by 100. The atomic percentage or atomic weight percent (awt. %) is the number of atoms of that element, at that weight percentage, divided by the total number of atoms in the sample multiplied by 100. The use of the terms weight percentage (wt. %) and atomic percentage or atomic weight percentage (awt. %) are two ways of referring to metallic alloy and its constituents. It has been found that for several metal alloys the inclusion of rhenium and/or hafnium results in the ductility and/or tensile strength of the metal alloy to improve as compared to a metal alloy is that absent rhenium and/or hafnium. Such improvement in ductility and/or tensile strength due to the inclusion of at least 5-15 awt. % rhenium and/or 5-15 awt. % hafnium in the metal alloy is referred to as the “rhenium effect” or “hafnium effect.” As defined herein, a “rhenium effect” or a “hafnium effect” is a) an increase of at least 10% in ductility of the metal alloy caused by the addition of rhenium and/or hafnium to the metal alloy, and/or b) an increase of at least 10% in tensile strength of the metal alloy caused by the addition of rhenium and/or hafnium to the metal alloy. As defined herein, a refractory metal alloy is a metal alloy that includes at least 20 wt. % of one or more of molybdenum, rhenium, niobium, tantalum or tungsten. Non-limiting refractory metal alloys include MoRe alloy, ReW alloy, MoReCr alloy, MoReTa alloy, MoReTi alloy, WCu alloy, ReCr, molybdenum alloy, rhenium alloy, tungsten alloy, tantalum alloy, niobium alloy, etc.

In accordance with another and/or alternative aspect of the present disclosure, the metal alloy used to partially or fully form the metal plate includes stainless-steel, CoCr alloys, TiAlV alloys, aluminum alloys, nickel alloys, titanium alloys, tungsten alloys, molybdenum alloys, copper alloys, MP35N alloys, or beryllium-copper alloys that have been modified to include at least 5-15 awt. % rhenium and/or 5-15 awt % hafnium so as to result in improved ductility and/or tensile strength as compared to the same metal alloy that is absent rhenium and/or hafnium. As defined herein, a stainless-steel alloy (SS alloy) includes at least 50 wt. % iron (e.g., 50-85 wt. % and all values and ranges therebetween), 10-30 wt. % chromium, 0-35 wt. % nickel, and optionally one or more of 0-5 wt. % molybdenum, 0-6 wt. % manganese, 0-1 wt. % silicon, 0-0.3 wt. % carbon, 0-5 wt. % titanium, 0-10 wt. % niobium, 0-5 wt. % copper, 0-4 wt. % aluminum, 0-10 wt. % tantalum, 0-1 wt. % Se, 0-2 wt. % vanadium, and 0-2 wt. % tungsten. A 316L alloy that falls within a stainless-steel alloy includes 17-19 wt. % chromium, 13-15 wt. % nickel, 2-4 wt. % molybdenum, 2 wt. % max manganese, 0.75 wt. % max silicon, 0.03 wt. % max carbon, balance iron. As defined herein, a cobalt-chromium alloy (CoCr alloy) includes 30-72 wt. % cobalt, 15-35 wt. % chromium, and optionally one or more of 1-38 wt. % nickel, 2-18 wt. % molybdenum, 0-18 wt. % iron, 0-1 wt. % titanium, 0-2.8 wt. % manganese, 0-0.15 wt. % silver, 0-2 wt. % carbon, 0-16 wt. % tungsten, 0-2 wt. % silicon, 0-2 wt. % aluminum, 0-1 wt. % iron, 0-0.1 wt. % boron, 0-0.15 wt. % silver, and 0-2 wt. % titanium. As a MP35N alloy that falls within a CoCr alloy includes 18-22 wt. % chromium, 32-38 wt. % nickel, 8-12 wt. % molybdenum, 0-2 wt. % iron, 0-0.5 wt. % silicon, 0-0.5 wt. % manganese, 0-0.2 wt. % carbon, 0-2 wt. % titanium, 0-0.1 wt. %, 0-0.1 wt. % boron, 0-0.15 wt. % silver, and balance cobalt. As defined herein, a Phynox and Elgiloy alloy that falls within a CoCr alloy includes 38-42 wt. % cobalt, 18-22 wt. % chromium, 14-18 wt. % iron, 13-17 wt. % nickel, 6-8 wt. % molybdenum. As defined herein, a L605 alloy that falls within a CoCr alloy includes 18-22 wt. % chromium, 14-16 wt. % tungsten, 9-11 wt. % nickel, balance cobalt. As defined herein, a titanium-aluminum-vanadium alloy (TiAlV alloy) includes 4-8 wt. % aluminum, 3-6 wt. % vanadium, 80-93 wt. % titanium, and optionally one or more of 0-0.4 wt. % iron, 0-0.2 wt. % carbon, 0-0.5 wt. % yttrium. A Ti-6Al-4V alloy that falls with a TiAlV alloy includes incudes 3.5-4.5 wt. % vanadium, 5.5-6.75 wt. % aluminum, 0.3 wt. % max iron, 0.08 wt. % max carbon, 0.05 wt. % max yttrium, balance titanium. As defined herein, an aluminum alloy includes 80-99 wt. % aluminum, and optionally one or more 0-12 wt. % silicon, 0-5 wt. % magnesium, 0-1 wt. % manganese, 0-0.5 wt. % scandium, 0-0.5 wt. % beryllium, 0-0.5 wt. % yttrium, 0-0.5 wt. % cerium, 0-0.5 wt. % chromium, 0-3 wt. % iron, 0-0.5, 0-9 wt. % zinc, 0-0.5 wt. % titanium, 0-3 wt. % lithium, 0-0.5 wt. % silver, 0-0.5 wt. % calcium, 0-0.5 wt. % zirconium, 0-1 wt. % lead, 0-0.5 wt. % cadmium, 0-0.05 wt. % bismuth, 0-1 wt. % nickel, 0-0.2 wt. % vanadium, 0-0.1 wt. % gallium, and 0-7 wt. % copper. As defined herein, a nickel alloy includes 30-98 wt. % nickel, and optionally one or more 5-25 wt. % chromium, 0-65 wt. % iron, 0-30 wt. % molybdenum, 0-32 wt. % copper, 0-32 wt. % cobalt, 2-2 wt. % aluminum, 0-6 wt. % tantalum, 0-15 wt. % tungsten, 0-5 wt. % titanium, 0-6 wt. % niobium, 0-3 wt. % silicon. As defined herein, a titanium alloy includes 80-99 wt. % titanium, and optionally one of more of 0-6 wt. % aluminum, 0-3 wt. % tin, 0-1 wt. % palladium, 0-8 wt. % vanadium, 0-15 wt. % molybdenum, 0-1 wt. % nickel, 0-0.3 wt. % ruthenium, 0-6 wt. % chromium, 0-4 wt. % zirconium, 0-4 wt. % niobium, 0-1 wt. % silicon, 0.0.5 wt. % cobalt, 0-2 wt. % iron. As defined herein, a tungsten alloy includes 85-98 wt. % tungsten, and optionally one or more of 0-8wt. % nickel, 0-5 wt. % copper, 0-5 wt. % molybdenum, 0-4 wt. % iron. As defined herein, a molybdenum alloy includes 90-99.5 wt. % molybdenum, and optionally one or more of 0-1 wt. % nickel, 0-1 wt. % titanium, 0-1 wt. % zirconium, 0-30 wt. % tungsten, 0-2 wt. % hafnium, 0-2 wt. % lanthanum. As defined herein, a copper alloy includes 55-95 wt. % copper, and optionally one or more of 0-40 wt. % zinc, 0-10 wt. % tin, 0-10 wt. % lead, 0-1 wt. % iron, 0-5 wt. % silicon, 0-12 wt. % manganese, 0-12 wt. % aluminum, 0-3 wt. % beryllium, 0-1 wt. % cobalt, 0-20 wt. % nickel. As defined herein, a beryllium-copper alloy includes 95-98.5 wt. % copper, 1-4 wt. % beryllium, and optionally one or more of 0-1 wt. % cobalt, and 0-0.5 wt. % silicon. As defined herein, a titanium-nickel alloy (e.g., Nitinol alloy) includes 42-58 wt. % nickel and 42-58 wt. % titanium.

In accordance with another and/or alternative aspect of the present disclosure, the metal alloy used to partially or fully form the metal plate includes at least 5 awt. % (e.g., 5-99 awt. % and all values and ranges therebetween) rhenium, and 0.1-96 wt. % (and all values and ranges therebetween) of one or more additives selected from the group of aluminum, boron, beryllium, bismuth, cadmium, calcium, cerium, chromium, cobalt, copper, gallium, gold, hafnium, iridium, iron, lanthanum, lithium, magnesium, manganese, molybdenum, nickel, niobium, osmium, palladium, platinum, rare earth metals, rhodium, ruthenium, scandium, silver, silicon, tantalum, technetium, tin, titanium, tungsten, vanadium, yttrium, zinc, and/or zirconium, and the metal alloy optionally includes 0-2 wt. % (and all values and ranges therebetween) of a combination of other components other than the additives (e.g., carbon, oxygen, phosphorous, sulfur, hydrogen, lead, nitrogen, etc.), and which metal alloy exhibits a rhenium effect. In one non-limiting embodiment, the metal alloy is a stainless-steel alloy that has been modified to include at least 5-15 awt. % rhenium and/or hafnium. In another and/or alternative non-limiting embodiment, the metal alloy is a cobalt-chromium alloy that has been modified to include at least 5-15 awt. % rhenium and/or hafnium. In another and/or alternative non-limiting embodiment, the metal alloy is a TiAlV alloy that has been modified to include at least 5-15 awt. % rhenium and/or hafnium. In another and/or alternative non-limiting embodiment, the metal alloy is an aluminum alloy that has been modified to include at least 5-15 awt. % rhenium and/or hafnium. In another and/or alternative non-limiting embodiment, the metal alloy is a nickel alloy that has been modified to include at least 5-15 awt. % rhenium and/or hafnium. In another and/or alternative non-limiting embodiment, the metal alloy is a titanium alloy that has been modified to include at least 5-15 awt. % rhenium and/or hafnium. In another and/or alternative non-limiting embodiment, the metal alloy is a tungsten alloy that has been modified to include at least 15 awt. % rhenium and/or hafnium. In another and/or alternative non-limiting embodiment, the metal alloy is a molybdenum alloy that has been modified to include at least 5-15 awt. % rhenium and/or hafnium. In another and/or alternative non-limiting embodiment, the metal alloy is a copper alloy that has been modified to include at least 5-15 awt. % rhenium and/or hafnium. In another and/or alternative non-limiting embodiment, the metal alloy is a beryllium-copper alloy that has been modified to include at least 5-15 awt. % rhenium and/or hafnium.

In accordance with another and/or alternative aspect of the present disclosure, the metal alloy used to partially or fully form the metal plate includes at least 5 awt. % (e.g., 5-99 awt. % and all values and ranges therebetween) hafnium, and 0.1-96 wt. % (and all values and ranges therebetween) of one or more additives selected from the group of aluminum, boron, beryllium, bismuth, cadmium, calcium, cerium, chromium, cobalt, copper, gallium, gold, iridium, iron, lanthanum, lithium, magnesium, manganese, molybdenum, nickel, niobium, osmium, palladium, platinum, rare earth metals, rhenium, rhodium, ruthenium, scandium, silver, silicon, tantalum, technetium, tin, titanium, tungsten, vanadium, yttrium, zinc, and/or zirconium, and the metal alloy optionally includes 0-2 wt. % (and all values and ranges therebetween) of a combination of other components other than the additives (e.g., carbon, oxygen, phosphorous, sulfur, hydrogen, lead, nitrogen, etc.), and which metal alloy exhibits a hafnium effect. In one non-limiting embodiment, the metal alloy is a stainless-steel alloy that has been modified to include at least 5-15 awt. % hafnium. In another and/or alternative non-limiting embodiment, the metal alloy is a cobalt chromium alloy that has been modified to include at least 5-15 awt. % hafnium. In another and/or alternative non-limiting embodiment, the metal alloy is a TiAlV alloy that has been modified to include at least 5-15 awt. % hafnium. In another and/or alternative non-limiting embodiment, the metal alloy is an aluminum alloy that has been modified to include at least 5-15 awt. % hafnium. In another and/or alternative non-limiting embodiment, the metal alloy is a nickel alloy that has been modified to include at least 5-15 awt. % hafnium. In another and/or alternative non-limiting embodiment, the metal alloy is a titanium alloy that has been modified to include at least 5-15 awt. % hafnium. In another and/or alternative non-limiting embodiment, the metal alloy is a tungsten alloy that has been modified to include at least 5-15 awt. % hafnium. In another and/or alternative non-limiting embodiment, the metal alloy is a molybdenum alloy that has been modified to include at least 5-15 awt. % hafnium. In another and/or alternative non-limiting embodiment, the metal alloy is a copper alloy that has been modified to include at least 5-15 awt. % hafnium. In another and/or alternative non-limiting embodiment, the metal alloy is a beryllium-copper alloy that has been modified to include at least 5-15 awt. % hafnium.

In accordance with another and/or alternative aspect of the present disclosure, the metal alloy used to partially or fully form the metal plate includes rhenium and/or hafnium, and molybdenum, and the weight percent of rhenium and/or hafnium in the metal alloy is optionally greater than the weight percent of molybdenum in the metal alloy, and the weight percent of one or more additive (e.g., aluminum, boron, beryllium, bismuth, cadmium, calcium, cerium, chromium, cobalt, copper, gallium, gold, hafnium, iridium, iron, lanthanum, lithium, magnesium, manganese, molybdenum, nickel, niobium, osmium, palladium, platinum, rare earth metals, rhodium, ruthenium, scandium, silver, silicon, tantalum, technetium, tin, titanium, tungsten, vanadium, yttrium, zinc, and/or zirconium) in the metal alloy is optionally greater that the weight percent of molybdenum in the metal alloy, and the metal alloy optionally includes 0-2 wt. % of a combination of other components other than the additives (e.g., carbon, oxygen, phosphorous, sulfur, hydrogen, lead, nitrogen, etc.). In one non-limiting embodiment, the metal alloy is fully formed of or includes rhenium and/or hafnium, and molybdenum, and the weight percent of rhenium and/or hafnium plus the combined weight percent of additives is greater than the weight percent of molybdenum, and the metal alloy optionally includes 0-2 wt. % of a combination of other components other than the additives (e.g., carbon, oxygen, phosphorous, sulfur, hydrogen, lead, nitrogen, etc.).

In accordance with another and/or alternative aspect of the present disclosure, the metal alloy used to partially or fully form the metal plate includes rhenium and/or hafnium, and molybdenum, and one or more additives selected from bismuth, niobium, tantalum, tungsten, titanium, vanadium, chromium, manganese, yttrium, zirconium, technetium, ruthenium, rhodium, hafnium, osmium, copper, and iridium, and the atomic weight percent of rhenium and/or hafnium to the atomic weight percent of the combination of one or more of bismuth, niobium, tantalum, tungsten, titanium, vanadium, chromium, manganese, yttrium, zirconium, technetium, ruthenium, rhodium, hafnium, osmium, copper, and iridium is 0.4:1 to 2.5:1 (and all values and ranges therebetween).

In accordance with another and/or alternative aspect of the present disclosure, the metal alloy used to partially or fully form the metal plate includes at least 5 awt. % (e.g., 5-99 awt. % and all values and ranges therebetween) rhenium and/or hafnium plus at least two metals selected from the group of molybdenum, bismuth, chromium, iridium, niobium, tantalum, titanium, yttrium, and zirconium, and the content of the metal alloy that includes other elements and compounds is 0-0.1 wt. %. In another and/or alternative non-limiting embodiment, the metal alloy includes rhenium and/or hafnium, molybdenum, and chromium. In another and/or alternative non-limiting embodiment, the metal alloy includes at least 35 wt. % (e.g., 35-75 wt. % and all values and ranges therebetween) rhenium and/or hafnium, and the metal alloy also includes chromium. In one non-limiting embodiment, the metal alloy includes at least 35 wt. % rhenium and/or hafnium, and at least 25 wt. % (e.g., 25-49.9 wt. % and all values and ranges therebetween) of the metal alloy includes chromium, and optionally 0.1-40 wt. % (and all values and ranges therebetween) of the metal alloy includes one or more of aluminum, boron, beryllium, bismuth, cadmium, calcium, cerium, chromium, cobalt, copper, gallium, gold, hafnium, iridium, iron, lanthanum, lithium, magnesium, manganese, molybdenum, nickel, niobium, osmium, palladium, platinum, rare earth metals, rhodium, ruthenium, scandium, silver, silicon, tantalum, technetium, tin, titanium, tungsten, vanadium, yttrium, zinc, and/or zirconium, and the metal alloy optionally includes 0-2 wt. % (and all values and ranges therebetween) of a combination of other metals, carbon, oxygen, phosphorous, sulfur, hydrogen and/or nitrogen. In another and/or alternative non-limiting embodiment, the metal alloy includes 15-50 awt. % rhenium and/or hafnium (and all values and ranges therebetween) and 0.5-70 awt. % chromium (and all values and ranges therebetween). In another and/or alternative non-limiting embodiment, the metal alloy includes 15-50 awt. % rhenium and/or hafnium (and all values and ranges therebetween) and 0.5-70 awt. % tantalum (and all values and ranges therebetween). In another and/or alternative non-limiting embodiment, the metal alloy includes 15-50 awt. % rhenium and/or hafnium (and all values and ranges therebetween) and 0.5-70 awt. % niobium (and all values and ranges therebetween). In another and/or alternative non-limiting embodiment, the metal alloy includes 15-50 awt. % rhenium and/or hafnium (and all values and ranges therebetween) and 0.5-70 awt. % titanium (and all values and ranges therebetween). In another and/or alternative non-limiting embodiment, the metal alloy includes 15-50 awt. % rhenium and/or hafnium (and all values and ranges therebetween) and 0.5-70 awt. % zirconium (and all values and ranges therebetween). In another and/or alternative non-limiting embodiment, the metal alloy includes 15-50 awt. % rhenium and/or hafnium (and all values and ranges therebetween) and 0.5-70 awt. % molybdenum (and all values and ranges therebetween). In another and/or alternative non-limiting embodiment, the metal alloy includes at least 15 awt. % rhenium and/or hafnium, greater than 50 wt. % titanium (e.g., 51-80 wt. % and all values and ranges therebetween), 15-45 wt. % (and all values and ranges therebetween) niobium, 0-10 wt. % (and all values and ranges therebetween) zirconium, 0-15 wt. % (and all values and ranges therebetween) tantalum, and 0-8 wt. % molybdenum (and all values and ranges therebetween).

In accordance with another and/or alternative aspect of the present disclosure, the metal alloy used to partially or fully form the metal plate includes a refractory metal alloy, and wherein the refractory metal alloy includes at least 20 wt. % of one or more of niobium, rhenium, tantalum, molybdenum or tungsten (e.g., 20-99.9 wt. % and all values and ranges therebetween), and 0.1-80 wt. % (and all values and ranges therebetween) of one or more of calcium, carbon, cerium oxide, chromium, cobalt, copper, gold, hafnium, iridium, iron, lanthanum, lanthanum oxide, magnesium, manganese, molybdenum, nickel, osmium, platinum, rare earth metals, rhodium, ruthenium, silver, tantalum, technetium, titanium, vanadium, yttrium, yttrium oxide, zinc, zirconium, zirconium oxide, and/or alloys of one or more of such components.

In accordance with another and/or alternative aspect of the present disclosure, the metal alloy used to partially or fully form the metal plate includes a refractory metal alloy, and wherein the refractory metal alloy includes at least 5 awt. % rhenium and/or hafnium, at least 20 wt. % of one or more of niobium, tantalum, molybdenum or tungsten (e.g., 20-99.9 wt. % and all values and ranges therebetween), and 0-80 wt. % (and all values and ranges therebetween) of one or more of calcium, carbon, cerium oxide, chromium, cobalt, copper, gold, hafnium, iridium, iron, lanthanum, lanthanum oxide, magnesium, manganese, molybdenum, nickel, osmium, platinum, rare earth metals, rhodium, ruthenium, silver, tantalum, technetium, titanium, vanadium, yttrium, yttrium oxide, zinc, zirconium, zirconium oxide, and/or alloys of one or more of such components.

In accordance with another and/or alternative aspect of the present disclosure, the metal alloy used to partially or fully form the metal plate includes a refractory metal alloy, and wherein the refractory metal alloy includes at least 20 wt. % of one or more of niobium, tantalum or tungsten, and wherein the refractory metal alloy includes 0-30 wt. % molybdenum (and all values and ranges therebetween), and wherein the refractory metal alloy includes at least 5 awt. % rhenium and/or hafnium (e.g., 5-80 awt. % rhenium and/or hafnium and all values and ranges therebetween), and wherein the refractory metal alloy includes and 0-80 wt. % (and all values and ranges therebetween) of one or more of calcium, carbon, cerium oxide, chromium, cobalt, copper, gold, hafnium, iridium, iron, lanthanum, lanthanum oxide, magnesium, manganese, molybdenum, nickel, osmium, platinum, rare earth metals, rhodium, ruthenium, silver, tantalum, technetium, titanium, vanadium, yttrium, yttrium oxide, zinc, zirconium, zirconium oxide, and/or alloys of one or more of such components.

In accordance with another and/or alternative aspect of the present disclosure, the metal alloy used to partially or fully form the metal plate includes at least 5 awt. % rhenium and/or hafnium (e.g., 5-99 awt. % rhenium and/or hafnium and all values and ranges therebetween), and at least 0.1 wt. % of one or more additive metals selected from aluminum, bismuth, chromium, cobalt, copper, hafnium, iridium, iron, magnesium, manganese, nickel, niobium, osmium, rhodium, ruthenium, silicon, silver, tantalum, technetium, titanium, tungsten, vanadium, yttrium, and zirconium, and wherein the metal alloy includes 0-30 wt. % molybdenum (and all values and ranges therebetween), and wherein a combined weight percent of rhenium and/or hafnium, and the additive metals is 70-100 wt. % (and all values and ranges therebetween).

In accordance with another and/or alternative aspect of the present disclosure, the metal alloy used to partially or fully form the metal plate includes stainless-steel that has been modified with at least 5 awt. % rhenium and/or hafnium (e.g., 5-50 awt. % rhenium and/or hafnium and all values and ranges therebetween), and wherein a combined weight percent of iron, chromium, nickel, tantalum, niobium, copper, manganese, aluminum, titanium, selenium, vanadium, tungsten, hafnium and rhenium is 70-100 wt. % (and all values and ranges therebetween).

In accordance with another and/or alternative aspect of the present disclosure, the metal alloy used to partially or fully form the metal plate includes cobalt-chromium alloy that has been modified with at least 5 awt. % rhenium and/or hafnium (e.g., 5-50 awt. % rhenium and/or hafnium and all values and ranges therebetween), and wherein a combined weight percent of cobalt, chromium, nickel, iron, titanium, manganese, silver, tungsten, silicon, aluminum, iron, boron, silver, titanium, hafnium, and rhenium is 70-100 wt. % (and all values and ranges therebetween).

In accordance with another and/or alternative aspect of the present disclosure, the metal alloy used to partially or fully form the metal plate includes titanium-aluminum-vanadium alloy that has been modified with at least 5 awt. % rhenium and/or hafnium (e.g., 5-50 awt. % rhenium and/or hafnium and all values and ranges therebetween), and wherein a combined weight percent of aluminum, vanadium, titanium, iron, yttrium, hafnium, and rhenium is 70-100 wt. % (and all values and ranges therebetween).

In accordance with another and/or alternative aspect of the present disclosure, the metal alloy used to partially or fully form the metal plate includes aluminum alloy that has been modified with at least 5 awt. % rhenium and/or hafnium (e.g., 5-50 awt. % rhenium and/or hafnium and all values and ranges therebetween), and wherein a combined weight percent of aluminum, silicon, magnesium, manganese, scandium, beryllium, yttrium, cerium, chromium, iron, zinc, titanium, lithium, silver, calcium, zirconium, cadmium, bismuth, nickel, vanadium, gallium, copper, hafnium, and rhenium is 70-100 wt. % (and all values and ranges therebetween).

In accordance with another and/or alternative aspect of the present disclosure, the metal alloy used to partially or fully form the metal plate includes nickel alloy that has been modified with at least 5 awt. % rhenium and/or hafnium (e.g., 5-50 awt. % rhenium and/or hafnium and all values and ranges therebetween), and wherein a combined weight percent of nickel, chromium, iron, copper, cobalt, aluminum, tantalum, tungsten, titanium, niobium, silicon, hafnium, and rhenium is 70-100 wt. % (and all values and ranges therebetween).

In accordance with another and/or alternative aspect of the present disclosure, the metal alloy used to partially or fully form the metal plate includes titanium alloy that has been modified with at least 5 awt. % rhenium and/or hafnium (e.g., 5-50 awt. % rhenium and/or hafnium and all values and ranges therebetween), and wherein a combined weight percent of titanium, aluminum, tin, palladium, vanadium, nickel, ruthenium, chromium, zirconium, niobium, silicon, cobalt, iron, hafnium, and rhenium is 70-100 wt. % (and all values and ranges therebetween).

In accordance with another and/or alternative aspect of the present disclosure, the metal alloy used to partially or fully form the metal plate includes tungsten alloy that has been modified with at least 5 awt. % rhenium and/or hafnium (e.g., 5-50 awt. % rhenium and/or hafnium and all values and ranges therebetween), and wherein a combined weight percent of tungsten, nickel, copper, iron, hafnium, and rhenium is 70-100 wt. % (and all values and ranges therebetween).

In accordance with another and/or alternative aspect of the present disclosure, the metal alloy used to partially or fully form the metal plate includes copper alloy that has been modified with at least 5 awt. % rhenium and/or hafnium (e.g., 5-50 awt. % rhenium and/or hafnium and all values and ranges therebetween), and wherein a combined weight percent of copper, zinc, tin, iron, silicon, manganese, aluminum, beryllium, cobalt, nickel, hafnium, and rhenium is 70-100 wt. % (and all values and ranges therebetween).

In accordance with another and/or alternative aspect of the present disclosure, the metal alloy used to partially or fully form the metal plate includes beryllium-copper alloy that has been modified with at least 5 awt. % rhenium and/or hafnium (e.g., 5-50 awt. % rhenium and/or hafnium and all values and ranges therebetween), and wherein a combined weight percent of copper, beryllium, cobalt, silicon, hafnium, and rhenium is 70-100 wt. % (and all values and ranges therebetween).

In accordance with another and/or alternative aspect of the present disclosure, the metal alloy used to partially or fully form the metal plate includes titanium-nickel alloy that has been modified with at least 5 awt. % rhenium and/or hafnium (e.g., 5-50 awt. % rhenium and/or hafnium and all values and ranges therebetween), and wherein a combined weight percent of nickel, titanium, hafnium, and rhenium is 70-100 wt. % (and all values and ranges therebetween).

In accordance with another and/or alternative aspect of the present disclosure, the metal alloy used to partially or fully form the metal plate includes a metal alloy that includes less than 5 wt. % nickel (e.g., 0-4.99 wt. % and all values and ranges therebetween).

In accordance with another and/or alternative aspect of the present disclosure, the metal alloy used to partially or fully form the metal plate includes a metal alloy that includes less than 5 wt. % chromium (e.g., 0-4.99 wt. % and all values and ranges therebetween).

In accordance with another and/or alternative aspect of the present disclosure, the metal alloy used to partially or fully form the metal plate includes a primary metal (5-95 wt. % primary metal and all values and ranges therebetween) selected from one or more of molybdenum, rhenium, hafnium, niobium, tantalum, tungsten, and one or more alloying agents such as, but are not limited to, calcium, carbon, cerium oxide, chromium, cobalt, copper, gold, hafnium, iron, lanthanum oxide, magnesium, nickel, osmium, platinum, rare earth metals, rhenium, silver, technetium, titanium, vanadium, yttrium, yttrium oxide, zinc, zirconium, zirconium oxide, and/or alloys of one or more of such components (e.g., MoHfC, MoY₂O₃, MoCs₂O, MoW, MoTa, MoZrO₂, MoLa₂O₃, MoRe alloy, MoReW alloy, HfMo alloy, HfW alloy, ReW alloy, etc.).

In accordance with another and/or alternative aspect of the present disclosure, the metal alloy used to partially or fully form the metal plate includes tungsten and copper and optionally one or more metal agents such as, but are not limited to, calcium, carbon, cerium oxide, chromium, cobalt, gold, hafnium, iron, lanthanum oxide, magnesium, molybdenum, nickel, niobium, osmium, platinum, rare earth metals, rhenium, silver, tantalum, technetium, titanium, vanadium, yttrium, yttrium oxide, zinc, zirconium, zirconium oxide, and/or alloys of one or more of such components. In one non-limiting formulation, the metal alloy includes 1-99.9 wt. % tungsten (and all values and ranges therebetween) (e.g., 1 wt. %, 1.01 wt. %, 1.02 wt. % . . . 99.88 wt. %, 99.89 wt. %, 99.9 wt. %), and 0.1-99 wt. % copper (and all values and ranges therebetween) (e.g., 0.1 wt. %, 0.101 wt. %, 0.102 wt. % . . . 98.998 wt. %, 98.999 wt. %, 99 wt. %). In another non-limiting formulation, the tungsten constitutes the greatest weight percent in the metal alloy and the copper constitutes the second greatest weight percent in the metal alloy. In another non-limiting formulation, the tungsten constitutes the largest weight percent of any component that forms the metal alloy. In another non-limiting formulation, the tungsten constitutes greater than 50 wt. % of the metal alloy.

In accordance with another and/or alternative aspect of the present disclosure, the metal alloy used to partially or fully form the metal plate includes tungsten and rhenium and/or hafnium, and optionally one or more alloying agents such as, but not limited to, calcium, carbon, cerium oxide, chromium, cobalt, copper, gold, hafnium, iron, lanthanum oxide, magnesium, molybdenum, nickel, niobium, osmium, platinum, rare earth metals, rhenium, silver, tantalum, technetium, titanium, tungsten, vanadium, yttrium, yttrium oxide, zinc, zirconium, zirconium oxide, and/or alloys of one or more of such components (e.g., WRe, WReMo, WHf, WHfMo, WHfRe, WHfReMo, etc.). In one non-limiting formulation, the metal alloy includes 1-40 wt. % rhenium and/or hafnium (and all values and ranges therebetween and 60-99 wt. % tungsten (and all values and ranges therebetween). The total weight percent of the tungsten, rhenium and hafnium in the metal alloy is at least about 95 wt. % (e.g., 95-100% and all values and ranges therebetween). In another non-limiting formulation, the metal alloy includes 1-47.5 wt. % rhenium and/or hafnium (and all values and ranges therebetween) and 20-80 wt. % tungsten (and all values and ranges therebetween) and 0-47.5 wt. % molybdenum (and all values and ranges therebetween). The total weight percent of the tungsten, molybdenum, rhenium and hafnium in the metal alloy is at least about 95 wt. % (e.g., 95-100% and all values and ranges therebetween). In one non-limiting specific metal alloy, the weight percent of the tungsten is greater than a weight percent of rhenium, hafnium and/or molybdenum. In another non-limiting specific metal alloy, the weight percent of the tungsten is greater than 50 wt. % of the metal alloy. In another non-limiting specific metal alloy, the weight percent of the tungsten is greater than a weight percent of rhenium and/or hafnium, but is less than a weigh percent of molybdenum. In another non-limiting specific metal alloy, the weight percent of the tungsten is greater than a weight percent of molybdenum, but less than a weigh percent of rhenium and/or hafnium. In another non-limiting metal alloy, the weight percent of the tungsten is less than a weight percent of rhenium, hafnium and/or molybdenum.

In accordance with another and/or alternative aspect of the present disclosure, the metal alloy used to partially or fully form the metal plate includes a metal alloy that has a atomic weight percent of rhenium and/or hafnium to the atomic weight percent of the combination of bismuth, niobium, tantalum, tungsten, titanium, vanadium, chromium, manganese, yttrium, zirconium, technetium, ruthenium, rhodium, hafnium, osmium, copper, and iridium in the metal alloy is 0.7:1 to 1.5:1 (and all values and ranges therebetween).

In accordance with another and/or alternative aspect of the present disclosure, the metal alloy used to partially or fully form the metal plate includes that includes two of bismuth, niobium, tantalum, tungsten, titanium, vanadium, chromium, manganese, yttrium, zirconium, technetium, ruthenium, rhodium osmium, copper, and iridium, the atomic ratio of the two metals is 0.4:1 to 2.5:1 (and all values and ranges therebetween).

In accordance with another and/or alternative aspect of the present disclosure, the metal alloy used to partially or fully form the metal plate includes a titanium-nickel alloy that has been modified with at least 5 awt. % rhenium and/or hafnium (e.g., 5-50 awt. % rhenium and/or hafnium and all values and ranges therebetween), and wherein a combined weight percent of nickel, titanium, hafnium, and rhenium is 70-100 wt. % (and all values and ranges therebetween).

In accordance with another and/or alternative non-limiting aspect of the present disclosure, at least 10 wt. % (e.g., 10-95 wt. % and all values and ranges therebetween) of the metal alloy that includes at least 15 atw. % hafnium and/or rhenium and also includes one or more of molybdenum, niobium, tantalum, or tungsten.

In accordance with another and/or alternative non-limiting aspect of the present disclosure, there is provided a metal alloy that includes at least 15 atw. % hafnium and/or rhenium and 0.1-75 wt. % (and all values and ranges therebetween) of one or more of aluminum, bismuth, calcium, carbon, chromium, cobalt, copper, gold, iridium, iron, lanthanum, magnesium, manganese, molybdenum, nickel, niobium, osmium, platinum, rare earth metals, rhodium, ruthenium, silver, tantalum, technetium, titanium, tungsten, vanadium, yttrium, zinc, zirconium, and/or alloys of one or more of such components.

In another non-limiting aspect of the present disclosure, the metals used to form the metal alloy includes at least 15 atw. % hafnium and/or rhenium, nickel and tungsten and optionally one or more alloying agents such as, but not limited to, aluminum, bismuth, calcium, carbon, chromium, cobalt, copper, gold, iron, magnesium, molybdenum, niobium, osmium, platinum, rare earth metals, rhenium, silver, tantalum, technetium, titanium, vanadium, yttrium, zinc, zirconium, and/or alloys of one or more of such components (e.g., WNi, WNiMo, WNi. Re, etc.). In another non-limiting formulation, the metal alloy that includes at least 15 atw. % hafnium and/or rhenium includes 1-47.5 wt. % nickel (and all values and ranges therebetween) and 20-80 wt. % tungsten (and all values and ranges therebetween) and 1-47.5 wt. % (and all values and ranges therebetween) of one or more of aluminum, bismuth, calcium, carbon, chromium, cobalt, copper, gold, hafnium, iron, magnesium, molybdenum, nickel, niobium, osmium, platinum, rare earth metals, silver, tantalum, technetium, titanium, vanadium, yttrium, zinc, zirconium.

In accordance with another and/or alternative non-limiting aspect of the present disclosure, the metal alloy includes at least 15 atw. % hafnium and/or rhenium and nickel.

In accordance with another and/or alternative aspect of the present disclosure, the metal alloy includes at least 15 atw. % hafnium and/or rhenium includes (e.g., 15-99 awt. % and all values and ranges therebetween), optionally 5 awt. % (e.g., 5-99 awt. % and all values and ranges therebetween) molybdenum, optionally 5 awt. % (e.g., 5-99 awt. % and all values and ranges therebetween) niobium, optionally 5 awt. % (e.g., 5-99 awt. % and all values and ranges therebetween) tantalum, optionally 5 awt. % (e.g., 5-99 awt. % and all values and ranges therebetween) tungsten, and at least 0.1 wt. % (e.g., 0.1 wt. % to 96 wt. % and all values and ranges therebetween) of one or more of aluminum, boron, beryllium, bismuth, cadmium, calcium, cerium, chromium, cobalt, copper, gallium, gold, iridium, iron, lanthanum, lithium, magnesium, manganese, nickel, osmium, palladium, platinum, rare earth metals, rhodium, ruthenium, scandium, silver, silicon, technetium, tin, titanium, vanadium, yttrium, zinc, and/or zirconium, and the metal alloy optionally includes 0-2 wt. % (and all values and ranges therebetween) of a combination of other metals, carbon, oxygen, phosphorous, sulfur, hydrogen and/or nitrogen.

In accordance with another and/or alternative non-limiting aspect of the present disclosure, there is provided a metal plate partially or fully formed of a metal alloy that includes at least 15 atw. % hafnium. In one non-limiting embodiment, 50-100% (and all values and ranges therebetween) of the medical device or other type of device is formed of the metal alloy that includes at least 15 atw. % hafnium. In another non-limiting embodiment, at least 30 wt. % (e.g., 30-100 wt. % and all values and ranges therebetween) of the medical device or other type of device is formed of a metal alloy that includes at least 15 atw. % hafnium.

In accordance with another and/or alternative aspect of the present disclosure, the metal plate is fully formed of or includes a metal alloy that includes less than 5 wt. % nickel (e.g., 0-4.99 wt. % and all values and ranges therebetween).

In accordance with another and/or alternative aspect of the present disclosure, the metal plate is fully formed of or includes a metal alloy that includes less than 5 wt. % chromium (e.g., 0-4.99 wt. % and all values and ranges therebetween).

In accordance with another and/or alternative aspect of the present disclosure, the metal plate is fully formed of or includes a metal alloy that has a reduced ion release when the metal plate is implanted in a patient as compared to a metal plate formed of a metal alloy that does not include at least 5-15 awt % rhenium and/or hafnium. In one non-limiting embodiment, the metal alloy that includes at least 5-15 awt % rhenium and/or hafnium has a maximum ion release of a primary component of the metal alloy when inserted or implanted on or in the body of the patient of no more than 0.5 μg/cm² per day (e.g., 0.001-0.5 μg/cm² per day and all values and ranges therebetween); and wherein the primary component is a component of the metal alloy that constitutes at least 2 wt. % of the metal alloy; and wherein the metal alloy optionally has an absolute increase in ion release per dose of metal alloy in the tissue about the implanted medical device of no more than 50 days (e.g., 5-50 days and all values and ranges therebetween) after inserted or implanted on or in the body of a patient; and wherein the metal alloy optionally has no more than 50% (e.g., 0-50% and all values and ranges therebetween) of the allowed daily exposure of primary metal form the metal alloy during the first 5 days after inserted or implanted on or in the body of a patient, and optionally has no more than 20% (e.g., 0-20% and all values and ranges therebetween) of the allowed daily exposure of primary metal form the metal alloy after the first 5 days after inserted or implanted on or in the body of a patient. As such, the metal alloy that includes at least 5 atw. % rhenium and/or hafnium or a refractory metal alloy optionally results in less potentially irritating metal ions (e.g., nickel ions, chromium ions, etc.) that are released from the metal alloy as compared to ion release from stainless steel, cobalt-chromium alloy, nickel-titanium alloy, or TiAlV alloy.

In accordance with another and/or alternative aspect of the present disclosure, the metal plate is fully formed of or includes a metal alloy that includes at least 5 awt. % rhenium and/or hafnium such that the metal alloy that has a greater hydrophilicity as compared to metal plates formed of metal alloy that does not include at least 5 awt. % rhenium and/or hafnium. In one non-limiting embodiment, the surface of a metal alloy that includes at least 5 awt. % rhenium and/or hafnium has a hydrophilicity wherein the contact angle of a water droplet on the surface of the metal alloy is 25°-45° (and all values and ranges therebetween), and typically 30-42°.

In accordance with another and/or alternative aspect of the present disclosure, there is optionally provided a near net process for forming a metal plate in accordance with the preset disclosure. In one non-limiting embodiment of the disclosure, there is provided a method of powder pressing materials and increasing the strength post-sintering, and optionally imparting additional cold work to the formed metal alloy (e.g., swaging, etc.). In one non-limiting embodiment, metal alloy powder is pressed and then sintered to form a green part. Thereafter, the green part can optionally again be pressed to increase its mechanical strength. Such additional pressing can be a swaging process or some other or additional pressing process.

In accordance with another and/or alternative aspect of the present disclosure, the medical device that is partially or fully formed from a metal plate in accordance with the present disclosure can optionally include the steps of initially forming a plate, and then finished the plate into final form of the medical device by one or more finishing processes. The metal plate can be formed by various techniques such as, but not limited to, 1) melting the metal alloy and/or metals that form the metal alloy (e.g., vacuum arc melting, etc.) and then extruding and/or casting the metal alloy into a plate or sheet of metal alloy, 2) consolidating the metal powder of the metal alloy and/or metal powder of metals that form a plate of metal alloy, or 3) 3-D printing of the metal plate from metal powder of the metal alloy and/or from metal powder of metals that form the metal alloy, and/or 3-D printing of the metal plate from a wire of metal alloy.

In accordance with another and/or alternative aspect of the present disclosure, when the metal powder is consolidated to form of a green plate that has the same or similar shape and size as the plate to be used to form the medical device. The metal powder is pressed together to form a solid solution of the metal alloy. Typically, the pressing process is by an isostatic process (i.e., uniform pressure applied from all sides on the metal powder); however other processes can be used. When the metal powders are pressed together isostatically, cold isostatic pressing (CIP) is typically used to consolidate the metal powders; however, this is not required. The pressing process can be performed in an inert atmosphere, an oxygen-reducing atmosphere (e.g., hydrogen, argon and hydrogen mixture, etc.), and/or under a vacuum; however, this is not required. In one non-limiting embodiment, the average particle size of the metal powders used to form the metal plate is less than about 230 mesh (e.g., less than 63 microns; 1-62 microns and all values and ranges therebetween). In another and/or alternative non-limiting embodiment, the average particle size of the metal powders is about 2-62 microns, and more particularly about 5-49.9 microns. In another and/or alternative non-limiting embodiment, the average particle size of the metal powders is about 10-40 microns. In another and/or alternative non-limiting embodiment, the average density of the metal powders used to form the metal plate is greater than 5 g/cm3 (e.g., 5.001 g/cm3 to 19.3 g/cm3 and all values and ranges therebetween). In another and/or alternative non-limiting embodiment, 10-100 vol. % (and all values and ranges therebetween) of the metal powder that is used to form the metal plate is spherical shaped. In another and/or alternative non-limiting embodiment, the purity of the metal powders used to form the metal plate contain low levels of carbon, oxygen, and nitrogen. Typically, the carbon content of the metal powder used to form the metal alloy is less than about 100 ppm, the oxygen content is less than about 50 ppm, and the nitrogen content is less than about 20 ppm. Typically, metal powder used to form the metal alloy has a purity grade of at least 99.9 and more typically at least about 99.95.

In accordance with another and/or alternative aspect of the present disclosure, when the metal powder is consolidated to form the metal alloy into a metal plate in accordance with the present disclosure, the pressing process used to consolidate the metal powder can be by an isostatic process (i.e., uniform pressure applied from all sides on the metal powder); however other processes can be used. When the metal powders are pressed together isostatically, cold isostatic pressing (CIP) is typically used to consolidate the metal powders; however, this is not required. The pressing process can be performed in an inert atmosphere, an oxygen-reducing atmosphere (e.g., hydrogen, argon and hydrogen mixture, etc.), and/or under a vacuum; however, this is not required. The average density of the near net metal that is obtained by pressing together the metal powders is about 80-95% (and all values and ranges therebetween) of the final average density of the metal plate, or about 70-96% (and all values and ranges therebetween) the minimum theoretical density of the metal alloy used to form the metal plate. Pressing pressures of at least about 300 MPa are generally used. Generally, the pressing pressure is about 400-700 MPa; however, other pressures can be used. After the metal powders are pressed together, the pressed metal powders are sintered to partially or fully fuse the metal powders together to form the green part or green metal plate. The sintering of the consolidated metal powder can optionally be performed in an oxygen-reduced atmosphere (e.g., helium, argon, hydrogen, argon and hydrogen mixture, etc.), and/or under a vacuum, and/or some other non-oxidizing environment. At the high sintering temperatures, a high hydrogen atmosphere will reduce both the amount of carbon and oxygen in the formed green part or green plate. The sintered metal powder generally has an as-sintered average density of about 90-99% the minimum theoretical density of the metal alloy that is used to form the metal plate.

In accordance with another and/or alternative aspect of the present disclosure, the metal alloy used to at least partially form the metal plate is formed from one or more ingots of metal or metal alloy. In one non-limiting process, an arc melting process (e.g., vacuum arc melting process, etc.) can be used to form the metal plate.

In accordance with another and/or alternative aspect of the present disclosure, the green part or green plate that is formed of the metal alloy is optionally further treated by a swaging process; however, this is not required. In one non-limiting embodiment, swaging is performed on the green part or green plate metal alloy to at least partially or fully achieve final dimensions of metal plate that is used to form the medical device. The swaging dies can be shaped to fit the final dimension of the metal plate; however, this is not required. The swaging operation can be used to harden the plate. Swaging temperatures can be from room temperature (RT) (e.g., 10-27° C. and all values and ranges therebetween) to about 1500° C. (e.g., 10-1500° C. and all values and ranges therebetween). For lower swaging temperatures (e.g., temperatures less than 400° C., the swaging can optionally be conducted in air or an oxidizing environment; however, a non-oxidizing environment can alternatively be used. For higher swaging temperatures, the swaging process typically occurs in a controlled neutral or non-reducing environment (e.g., inert environment, non-oxidizing environment, vacuum, etc.). In one non-limiting embodiment, during the swaging process ions of boron and/or nitrogen are allowed to impinge upon rhenium and/or hafnium atoms in the metal alloy that includes rhenium and/or hafnium so as to form ReB₂, ReN₂, ReN₃, HfB₂, HfN₂ and/or HfN₃; however, this is not required. It has been found that ReB₂, ReN₂, ReN₃, HfB₂, HfN₂ and/or HfN3 are ultra-hard compounds.

In accordance with another and/or alternative aspect of the present disclosure, the metal plate can be optionally machined, cut and/or shaped when the metal plate is in a less hardened state. As such, the metal plate can optionally annealed to soften the metal plate and then machine and/or cut into a desired shape. After the metal plate has been optionally machined, cut and/or shaped, the metal plate can optionally be re-hardened (e.g., subjecting the metal plate to a swaging process, etc.).

In accordance with another and/or alternative aspect of the present disclosure, the metal plate that is partially or fully formed of the metal alloy can optionally be nitrided; however, this is not required. The nitrided layer on the top surface of the metal alloy can function as a lubricating surface or a protecting surface. After the metal alloy is nitrided, the metal alloy is typically cleaned; however, this is not required. During the nitriding process, the surface of the metal alloy is modified by the presence of nitrogen. The nitriding process can be by gas nitriding, salt bath nitriding, or plasma nitriding. In gas nitriding, the nitrogen diffuses onto the surface of the metal alloy, thereby creating a nitrided layer. The thickness and phase constitution of the resulting nitriding layers can be selected and the process optimized for the particular properties required. The metal alloy can optionally be exposed to argon and/or hydrogen gas prior to the nitriding process to clean and/or preheat the metal alloy. These gases can be optionally used to clean oxide layers and/or solvents from the surface of the metal alloy. During the nitriding process, the metal alloy can optionally be exposed to hydrogen gas to inhibit or prevent the formation of oxides on the surface of the metal alloy. The thickness of the nitrided surface layer is less than about 1 mm. In one non-limiting embodiment, the thickness of the nitride surface layer is at least about 50 nanometer and less than about 1 mm (and all values and ranges therebetween). In another and/or alternative non-limiting embodiment, the thickness of the nitrided surface layer is at least about 50 nanometer and less than about 0.1 mm. Generally, the weight percent of nitrogen in the nitrided surface layer is 0.0001-5 wt. % nitrogen (and all values and ranges therebetween). In one non-limiting embodiment, the weight percent of nitrogen in the nitrided surface layer is generally less than one of the primary components of the metal alloy, and typically less than each of the two primary components of the metal alloy. For example, when a metal alloy is nitrided, the weight percent of the nitrogen in the nitrided surface layer is less than a weight percent of the rhenium and/or hafnium in the nitrided surface layer. In one non-limiting composition of the nitrided surface layer on a metal alloy (e.g., 47-55 wt. % rhenium and/or hafnium, 0.1-63 wt. % additional metal alloying agent), the nitrided surface layer comprises at least 30 wt. % rhenium and/or hafnium, and 0.0001-5 wt. % nitrogen (and all values and ranges therebetween). The nitriding process for the metal alloy can be used to increase surface hardness and/or wear resistance of the metal plate, and/or to inhibit or prevent discoloration of the metal alloy (e.g., discoloration by oxidation, etc.).

In accordance with another and/or alternative aspect of the present disclosure, the metal plate is cut and/or machined using one or more processes (e.g., water jet cutting, CNC machining, electrochemical etching, grinding, gun drilling, shearing, etc.) to partially (e.g., form 60-99.99% of one or more of the medical devices from the metal plate and all values and ranges therebetween) or fully form one or more medical devices from the metal plate. When two or more medical devices are cut and/or machined on the metal plate, the shape, size and/or configuration of the two of more medical devices can be the same or different. In one non-limiting embodiment, a water jet cutting apparatus can be used to cut 90-100% (and all values and ranges therebetween) of one or more medical devices on the metal plate. In one specific aspect, a water jet cutting apparatus is used to cut 92-99.9% of one or more medical devices on the metal plate. In another non-limiting aspect, the water jet cutting apparatus leaves two or more connection points between the one or more medical devices and the plate so that the one or more cut medical devices remain connected to the metal plate; however, this is not required. The water jet cutting device can be optionally used to smooth and/or round-off one or more of the outer edges on the outer perimeter of the one or more medical devices, and/or smooth and/or round-off the inner edges of one or more openings or slots in the one or more medical devices, which one or more opens are slots are spaced from the outer perimeter of the one or more medical devices; however, this is not required. As can be appreciated, other or additional process can be used to smooth and/or round-off the edges (e.g., polishing, grinding, etc.). In another and/or alternative non-limiting embodiment, a CNC process is used to cut 90-100% (and all values and ranges therebetween) of one or more medical devices on the metal plate. In one specific aspect, a CNC process is used to cut 92-99.9% of one or more medical devices on the metal plate. In another non-limiting aspect, the CNC process leaves two or more connection points between the one or more medical devices and the metal plate so that the one or more cut medical devices remain connected to the native plate; however, this is not required. The CNC process can be optionally used to smooth and/or round-off one or more of the outer edges on the outer perimeter of the one or more medical devices, and/or smooth and/or round-off the inner edges of one or more openings or slots in the one or more medical devices, which one or more opens are slots are spaced from the outer perimeter of the one or more medical devices; however, this is not required. As can be appreciated, other or additional process can be used to smooth and/or round-off the edges (e.g., polishing, grinding, water etching, etc.). In another and/or alternative non-limiting embodiment, electrochemical etching is used to cut 90-100% (and all values and ranges therebetween) of one or more medical devices on the metal plate. In one specific aspect, electrochemical etching is used to cut 92-99.9% of one or more medical devices on the metal plate. In another non-limiting aspect, the electrochemical etching leaves two or more connection points between the one or more medical devices and the metal plate so that the cut one or more medical devices remain connected to the metal plate; however, this is not required. The electrochemical etching can be optionally used to smooth and/or round-off one or more of the outer edges on the outer perimeter of the one or more medical devices, and/or smooth and/or round-off the inner edges of one or more openings or slots in the one or more medical devices, which one or more opens are slots are spaced from the outer perimeter of the one or more medical devices. As can be appreciated, other or additional process can be used to smooth and/or round-off the edges (e.g., polishing, grinding, water etching, etc.).

In accordance with another and/or alternative aspect of the present disclosure, when one or more medical devices are formed in the metal plate, the one or more medical devices can optionally include one or more connection points or regions such that the one or more medical devices remain connected to the metal plate. When one or more of the medical device remain connected to the metal plate, a non-limiting process or method for separating the one or more medical devices from the metal plate incudes cutting the one or more medical devices from the metal plate, shearing the one or more medical devices from the metal plate, grinding off one or more medical devices from the metal plate, etc. After the medical device is disconnected from the metal plate, the regions at and/or about the one or more connection points can be polished, smoothed, etc. to remove sharp surfaces and/or to form a desired shape/profile of the metal surface at or about the one or more connection points.

In accordance with another and/or alternative aspect of the present disclosure, one or more edges of the formed medical device can optionally be smoothed (e.g., smoothing the outer perimeter edges of the medical device, smoothing the edges of one or more openings that are spaced from the perimeter edge of the medical device, etc.) by use of one or more processes (e.g., polishing, grinding, articulating water jet, etc.). In one non-limiting embodiment, a CNC process is used to form the one or more medical devices in the metal plate, and thereafter a water jet process is used to smooth the edges on the formed medical devices. As can be appreciated, a polishing and/or grinding process can also or alternatively be used to smooth the edges on the one or more formed medical devices.

In accordance with another and/or alternative aspect of the present disclosure, after the formed medical device is separated from the metal plate, one or more regions of the formed medical device can optionally be bent to form the final shape of the medical device. Such bending can be accomplished by one or more processes (e.g., bending by use of a hydraulic press, bending by use of mating dies, etc.). In one non-limiting embodiment, a hydraulic press can be optionally used to bend one or more portions of the plate to formed the final or near final shape of the medical device. In another non-limiting embodiment, mating dies can be optionally used to facilitate in the bending of the formed medical device.

In another and/or alternative non-limiting aspect of the present disclosure, the formed medical device can include, contain, and/or be coated with one or more agents that facilitate in the success of the formed medical device and/or treated area. The term “agent” includes, but is not limited to a substance, pharmaceutical, biologic, veterinary product, drug, and analogs or derivatives otherwise formulated and/or designed to prevent, inhibit and/or treat one or more clinical and/or biological events, and/or to promote healing. The type and/or amount of an agent included in a formed medical device and/or coated on formed medical device can vary. When two or more agents are included in and/or coated on formed medical device, the amount of two or more agents can be the same or different. The type and/or amount of agent included on, in and/or in conjunction with formed medical device are generally selected to address one or more clinical events. Typically, the amount of agent included on, in, and/or used in conjunction with the formed medical device is about 0.01-100 ug per mm² and/or at least about 0.00001 wt. % of the device; however, other amounts can be used. In one non-limiting embodiment of the disclosure, the formed medical device can be partially or fully coated and/or impregnated with one or more agents to facilitate in the success of a particular medical procedure.

In a further and/or alternative non-limiting aspect of the present disclosure, the one or more agents on and/or in the formed medical device (when used) can be released in a controlled manner to provide the area in question to be treated with the desired dosage of agent over a sustained period of time. The formed medical device can be designed such that 1) all the agent on and/or in the formed medical device is controllably released, 2) some of the agent on and/or in the formed medical device is controllably released and some of the agent on the formed medical device is non-controllably released, or 3) none of the agent on and/or in the formed medical device is controllably released. The formed medical device can also be designed such that the rate of release of the one or more agents from the formed medical device is the same or different. The formed medical device can also be designed such that the rate of release of the one or more agents from one or more regions on the formed medical device is the same or different. Non-limiting arrangements that can be used to control the release of one or more agents from the formed medical device include 1) at least partially coating one or more agents with one or more polymers, 2) at least partially incorporating and/or at least partially encapsulating one or more agents into and/or with one or more polymers, and/or 3) inserting one or more agents in pores, passageway, cavities, etc., in the formed medical device and at least partially coating or covering such pores, passageway, cavities, etc., with one or more polymers. As can be appreciated, other or additional arrangements can be used to control the release of one or more agents from the formed medical device. The thickness of each polymer layer and/or layer of agent is generally at least about 0.01 μm and is generally less than about 150 μm (e.g., 0.01-149.9999 μm and all values and ranges therebetween). In one non-limiting embodiment, the thickness of a polymer layer and/or layer of agent is about 0.02-75 μm, more particularly about 0.05-50 μm, and even more particularly about 1-30 μm.

In yet another and/or alternative non-limiting aspect of the disclosure, the formed medical device can include a marker material. The marker material is typically designed to be visible to electromagnetic waves (e.g., x-rays, microwaves, visible light, infrared waves, ultraviolet waves, etc.); sound waves (e.g., ultrasound waves, etc.); magnetic waves (e.g., MRI, etc.), and/or other types of electromagnetic waves (e.g., microwaves, visible light, infrared waves, ultraviolet waves, etc.). In one non-limiting embodiment, the marker material is visible to x-rays (i.e., radiopaque). The marker material can form all or a portion of the formed medical device and/or be coated on one or more portions (flaring portion and/or body portion, at ends of formed medical device, at or near transition of body portion and flaring section, etc.) of the formed medical device. The location of the marker material can be on one or multiple locations on the formed medical device. The size of the one or more regions that include the marker material can be the same or different.

In a further and/or alternative non-limiting aspect of the present disclosure, the formed medical device or one or more regions of the formed medical device can be constructed by use of one or more microelectromechanical manufacturing (MEMS) techniques (e.g., micro-machining, laser micro-machining, laser micro-machining, micro-molding, etc.); however, other or additional manufacturing techniques can be used.

In accordance with another and/or alternative non-limiting aspect of the present disclosure, the formed medical device can be partially (e.g., 1% to 99.99% and all values and ranges therebetween) or fully be coated with an enhancement layer to improve one or more properties of the formed medical device (e.g., change exterior color of material having coated surface, increase surface hardness by use of the coated surface, increase surface toughness material having coated surface, reduced friction via use of the coated surface, improve scratch resistance of material that has the coated surface, improve impact wear of coated surface, improve resistance to corrosion and oxidation of coated material, form a non-stick coated surface, improve biocompatibility of material having the coated surface, reduce toxicity of material having the coated surface, reduce ion release from material having the coated surface, the enhancement layer forms a surface that is less of an irritant to cell about the coated surface after the formed medical device is implanted, reduces the rate to which cells grown on coated surface after the formed medical device is implanted, reduce rate to which movable components in the formed medical device fail to properly operate after orthopedic device is implanted, etc.).

In accordance with another and/or alternative non-limiting aspect of the present disclosure, non-limiting enhancement layers that can be applied to a portion or all of the outer surface of the formed medical device includes chromium nitride (CrN), diamond-like carbon (DLC), titanium nitride (TiN), titanium oxynitride or titanium nitride oxide (TiNOx), zirconium nitride (ZrN), zirconium oxide (ZrO₂), zirconium oxynitride (ZrNxOy) [e.g., cubic ZrN:O, cubic ZrO₂:N, tetragonal ZrO₂:N, and monoclinic ZrO₂:N phase coatings], oxyzirconium-nitrogen-carbon (ZrNC), zirconium OxyCarbide (ZrOC), and combinations of such coatings. The one or more enhancement layers are not considered an agent as defined in the present disclosure. The enhancement layer has a different composition from an agent as defined in the present disclosure. In one non-limiting embodiment, the one or more enhancement layers are optionally applied to a portion or all of the outer surface of the formed medical device by a vacuum process using an energy source to vaporize material and deposit a thin layer of enhancement layer material. Such vacuum coating process, when used, can include 1a physical vapor deposition (PVD) process (e.g., sputter deposition, cathodic arc deposition or electron beam heating, etc.), chemical vapor deposition (CVD) process, atomic layer deposition (ALD) process, or a plasma-enhanced chemical vapor deposition (PE-CVD) process. In one non-limiting embodiment, the coating process is one or more of a PVD, CVD, ALD and PE-CVD, and wherein the coating process occurs at a temperature of 200-400° C. (and all values and ranges therebetween) for at least 10 minutes (e.g., 10-400 minutes and all values and ranges therebetween). In another and/or alternative non-limiting embodiment, the coating process is one or more of a PVD, CVD, ALD and PE-CVD, and wherein the coating process occurs at a temperature of 220-300° C. for 60-120 minutes. In another and/or alternative non-limiting embodiment, when the materials of the one or more enhancement layers are to be applied to the outer surface of the formed medical device, the materials of the one or more enhancement layers can optionally be combine with one or more metals in the metal alloy, and/or combined with nitrogen, oxygen, carbon, or other elements that are in the metal alloy and/or present in the atmosphere about the metal alloy to a form an enhancement layer on the outer surface of the metal alloy. In another and/or alternative non-limiting embodiment, when the materials of the one or more enhancement layers are to be applied to the outer surface of the formed medical device, the materials of the one or more enhancement layers can optionally be used to form various coating colors on the outer surface of the metal alloy (e.g., gold, copper, brass, black, rose gold, chrome, blue, silver, yellow, green, etc.). In another and/or alternative non-limiting embodiment, the thickness of the enhancement layer is greater than 1 nanometer (e.g., 2 nanometers to 100 microns and all values and ranges therebetween), and typically 0.1-25 microns, and more typically 0.2-10 microns. In another and/or alternative non-limiting embodiment, the hardness of the enhancement layer can be at least 5 GPa (ASTM C 1327-15 or ASTM C 1624-05), typically 5-50 GPa (and all values and ranges therebetween), more typically 10-25 GPa, and still more typically 14-24 GPa. In another and/or alternative non-limiting embodiment, the coefficient of friction (COF) of the enhancement layer can be 0.04-0.2 (and all values and ranges therebetween), and typically 0.6-0.15. In another and/or alternative non-limiting embodiment, the wear rate of the enhancement layer can be 0.5×10⁻⁷ mm³/N-m to 3×10⁻⁷ mm³/N-m (an all values and ranges therebetween), and typically 1.2×10⁻⁷ mm³/N-m to 2×10⁻⁷ mm³/N-m. In another and/or alternative non-limiting embodiment, silicon-based precursors (e.g., trimethysilane, tetramethylsilane, hexachlorodisilane, silane, dichlorosilane, trichlorosilane, silicon tetrachloride, tris(dimethylamino) silane, bis(tert-butylamino)silane, trisilylamine, allyltrimethoxysilane, (3-aminopropyl)triethoxysilane, butyltrichlorosilane, n-sec-butyl(trimethylsilyl)amine, chloropentamethyldisilane, 1,2-dichlorotetramethyldisilane, [3-(diethylamino)propyl]trimethoxysilane, 1,3-diethyl-1,1,3,3-tetramethyldisilazane, dimethoxydimethylsilane, dodecamethylcyclohexasilane, hexamethyldisilane, isobutyl(trimethoxy)silane, methyltrichlorosilane, 2,4,6,8,10-pentamethylcyclopentasiloxane, pentamethyldisilane, n-propyltriethoxysilane, silicon tetrabromide, silicon tetrabromide, etc.) can optionally be used to facilitate in the application of the enhancement layer to one or more portions or all of the formed medical device. In one non-limiting embodiment, the enhancement layer includes no more than 0.1 wt. % nickel, no more than 0.1 wt. % chromium, and/or no more than 0.1 wt. % cobalt. In another and/or alternative non-limiting embodiment, the outer surface of the metal portion of the formed medical device includes no more than 0.1 wt. % nickel, no more than 0.1 wt. % chromium, and/or no more than 0.1 wt. % cobalt. The adhesion layer, when used, includes no more than 0.1 wt. % nickel, no more than 0.1 wt. % chromium, and/or no more than 0.1 wt. % cobalt.

In accordance with another and/or alternative non-limiting aspect of the present disclosure, one or more portions and/or components of the formed medical device can be partially or fully coated with an enhancement layer composition that includes a chromium nitride (CrN) coating. A portion or all of the formed medical device can be partially or fully coated with the chromium nitride (CrN) coating. The enhancement layer can be used to improve hardness, improve toughness, reduced friction, resistant impact wear, improve resistance to corrosion and oxidation, and/or form a reduced stick surface when in contact with many different materials. In accordance with one non-limiting embodiment, the chromium nitride (CrN) coating generally includes 40-85 wt. % Cr (and all values and ranges therebetween), 15-60 wt. % N (and all values and ranges therebetween), 0-10 wt. % Re (and all values and ranges therebetween), 0-10 wt. % Si (and all values and ranges therebetween), 0-2 wt. % O (and all values and ranges therebetween), and 0-2 wt. % C (and all values and ranges therebetween). In one non-limiting coating process, one or more portions and/or components of the formed medical device are initially coated with Cr metal. The Cr metal coating can be applied by PVD, CVD, ALD and PE-CVD in an inert environment. The coating thickness of Cr metal is 0.5-15 microns. Thereafter, the Cr metal coating is exposed to nitrogen gas and/or a nitrogen containing gas compound to cause the nitrogen to react with the Cr metal coating to form a layer of CrN on the outer surface of the Cr metal coating and/or the outer surface of one or more components of the formed medical device. Particles of Cr metal can optionally be mixed with nitrogen gas and/or a nitrogen containing gas compound to facilitate in the formation of the CrN coating. When Cr metal particles are used, the initial Cr coating layer on the outer surface of one or more components of the formed medical device can optionally be eliminated. In another and/or alternative non-limiting embodiment, the enhancement layer composition generally includes 65-80 wt. % Cr, 15-30 wt. % N, 0-8 wt. % Re, 0-1 wt. % Si, 0-1 wt. % O, and 0-1 wt. % C.

In accordance with another and/or alternative non-limiting aspect of the present disclosure, one or more portions and/or components of the formed medical device can be partially or fully coated with an enhancement layer composition that includes a diamond-Like Carbon (DLC) coating. A portion or all of the formed medical device can be partially or fully coated with the diamond-Like Carbon (DLC) coating. The enhancement layer can be used to improve hardness, improve toughness, reduced friction, resistant impact wear, improve resistance to corrosion and oxidation, improve biocompatibility, and/or form a reduced stick surface when in contact with many different materials. In one non-limiting embodiment, the diamond-Like Carbon (DLC) coating generally includes 60-99.99 wt. % C (and all values and ranges therebetween), 0-2 wt. % N (and all values and ranges therebetween), 0-10 wt. % Re (and all values and ranges therebetween), 0-20 wt. % Si (and all values and ranges therebetween), and 0-2 wt. % O (and all values and ranges therebetween). The carbon coating can be applied by PVD, CVD, ALD and PE-CVD in an inert environment. The carbon layer can be applied by use of methane and/or acetylene gas; however, other or additional carbon sources can be used. The coating thickness of the carbon is 0.5-15 microns. In another and/or alternative non-limiting embodiment, one or more portions and/or components of the formed medical device are coated with the enhancement layer composition that generally includes 90-99.99 wt. % C, 0-1 wt. % N, 0-8 wt. % Re, 0-1 wt. % Si, and 0-1 wt. % O.

In accordance with another and/or alternative non-limiting aspect of the present disclosure, one or more portions and/or components of the formed medical device can be partially or fully coated with an enhancement layer composition that includes a titanium nitride (TiN) coating. A portion or all of the outer surface of the formed medical device can include the titanium nitride (TiN) coating. The enhancement layer can be used to improve hardness, improve toughness, improve resistance to corrosion and oxidation, reduced friction, and/or form a reduced stick surface when in contact with many different materials. In one non-limiting embodiment, one or more portions and/or components of the formed medical device are optionally initially coated with Ti metal. The Ti metal coating, when applied, can be applied by PVD, CVD, ALD and PE-CVD in an inert environment. The coating thickness of Ti metal is 0.05-15 microns (and all values and ranges therebetween). As can be appreciated, the initial Ti coating is optional. Thereafter, the Ti metal coating, when applied, is exposed to nitrogen gas and/or a nitrogen containing gas compound and optionally titanium particles to cause the nitrogen to react with the Ti metal coating and/or titanium metal particles to form a layer of TiN on the outer surface of the Ti metal coating and/or the outer surface of the one or more portions or components of the formed medical device. If a titanium layer is not preapplied, the TiN coating can be formed by exposing the outer surface of one or more portions or components of the formed medical device to titanium particles and nitrogen gas and/or a nitrogen containing gas compound. The coating thickness of the TiN coating is generally at least 0.1 microns (e.g., 0.1-15 microns and all values and ranges therebetween), and typically 0.2-2 microns.

In accordance with another and/or alternative non-limiting aspect of the present disclosure, one or more portions and/or components of the formed medical device can be partially or fully coated with an enhancement layer composition that includes a titanium oxynitride or titanium nitride oxide (TiNOx) coating. A portion or all of the outer surface of the one or more portions or components of the formed medical device can include the titanium oxynitride or titanium nitride oxide (TiNOx) coating. The enhancement layer can be used to improve hardness, improve toughness, improve resistance to corrosion and oxidation, reduced friction, and/or form a reduced stick surface when in contact with many different materials, and/or promote nitric oxide formation on the surface of the coating. In one non-limiting embodiment, all or a portion of the outer surface of the formed medical device is optionally initially coated with Ti metal. The Ti metal coating, when applied, can be applied by PVD, CVD, ALD and PE-CVD in an inert environment. The coating thickness of Ti metal is 0.05-15 microns (and all values and ranges therebetween). As can be appreciated, the initial Ti coating is optional. Thereafter, the Ti metal coating is exposed to titanium particles and a nitrogen and oxygen mixture that can include nitrogen gas, oxygen gas, a nitrogen containing gas compound and/or an oxygen containing gas compound to cause the nitrogen and oxygen to react with the Ti metal coating, if such coating is used, and/or with the Ti metal particles to form a layer of TiNOx on the outer surface of the Ti metal coating and/or the outer surface of one or more portions or components of the formed medical device. The ratio of the N to the O can be varied to control the amount of O in the TiNOx coating. If a titanium layer is not preapplied, the TiNOx coating can be formed by exposing one or more portions or components of the formed medical device to titanium particles and a nitrogen and oxygen source such as nitrogen gas, oxygen gas, a nitrogen containing gas compound and/or an oxygen containing gas compound. The ratio of N to O when forming the TiNOx coating is generally 1:10 to 10:1 (and all values and ranges therebetween). The coating thickness of the TiNOx coating is generally at least 0.1 microns (e.g., 0.1-15 microns and all values and ranges therebetween), and typically 0.2-2 microns. In another and/or alternative non-limiting embodiment, a TiNOx coating is applied to a portion or all of the outer surface of the one or more portions or components of the formed medical device, and the TiNOx coating is formed by a) exposing the outer surface of a portion of all of the one or more portions or components of the formed medical device to Ti particles (PVD, CVD, ALD and PE-CVD process) and/or a Ti containing solution to form a Ti layer on a portion of all of the one or more portions or components of the formed medical device, and wherein the thickness of the Ti coating is 0.05-5 microns, and b) exposing the Ti coating to a nitrogen and oxygen source such as nitrogen gas, oxygen gas, a nitrogen containing gas compound and/or an oxygen containing gas compound to form a TiNOx coating, and wherein ratio of N to O when forming the TiNOx coating is generally 1:10 to 10:1, and wherein the coating thickness of the TiNOx coating is 0.2-5 microns. In another and/or alternative non-limiting embodiment, a TiNOx coating is applied to a portion or all of the outer surface of the one or more portions or components of the formed medical device, and the TiNOx coating is formed by exposing a portion or all of the outer surface of the formed medical device to Ti particles and a nitrogen and oxygen source such as nitrogen gas, oxygen gas, a nitrogen containing gas compound and/or an oxygen containing gas compound to form a TiNOx coating, and wherein ratio of N to O when forming the TiNOx coating is generally 1:10 to 10:1, and wherein the coating thickness of the TiNOx coating is 0.2-5 microns. In another and/or alternative non-limiting embodiment, the enhancement layer composition generally includes 20-85 wt. % Ti (and all values and ranges therebetween), 0.5-35 wt. % N (and all values and ranges therebetween), 0-10 wt. % Re (and all values and ranges therebetween), and 0.5-35 wt. % O (and all values and ranges therebetween). In another and/or alternative non-limiting embodiment, a coating of TiNOx was formed on one or more portions or components of the formed medical device by reactive physical vapor deposition in a vacuum chamber. Depending on the oxygen-nitrogen ratio during vapor deposition, a coating deposit of TiNOx with defined composition and resistivity can be coated on the outer surface of the one or more portions or components of the formed medical device.

In accordance with another and/or alternative non-limiting aspect of the present disclosure, one or more portions and/or components of the formed medical device can be partially or fully coated with an enhancement layer composition that includes a zirconium nitride (ZrN) coating. The enhancement layer can be used to improve hardness, improve toughness, improve resistance to corrosion and oxidation, reduced friction, and/or form a reduced stick surface when in contact with many different materials. In one non-limiting embodiment all or a portion of the outer surface of the one or more portions or components of the formed medical device is initially coated with Zr metal. The Zr metal coating can be applied by PVD, CVD, ALD and PE-CVD in an inert environment. The coating thickness of Zr metal is 0.5-15 microns. Thereafter, the Zr metal coating is exposed to nitrogen gas and/or a nitrogen containing gas compound to cause the nitrogen to react with the Zn metal coating to form a layer of ZrN on the outer surface of the Zr metal coating and/or the outer surface of one or more portions or components of the formed medical device. Particles of Zr metal can optionally be mixed with nitrogen gas and/or a nitrogen containing gas compound to facilitate in the formation of the ZrN coating. When Zr metal particles are used, the initial Zr coating layer on the outer surface of one or more portions or components of the formed medical device can optionally be eliminated. The ZrN coating has been found to produce a gold-colored enhancement layer color. In another and/or alternative non-limiting embodiment, the enhancement layer composition generally includes 35-90 wt. % Zr (and all values and ranges therebetween), 5-25 wt. % N (and all values and ranges therebetween), 0-10 wt. % Re (and all values and ranges therebetween), 0-20 wt. % Si (and all values and ranges therebetween), 0-2 wt. % O (and all values and ranges therebetween), and 0-2 wt. % C (and all values and ranges therebetween). In another and/or alternative non-limiting embodiment, the enhancement layer composition generally includes 80-90 wt. % Zr, 10-20 wt. % N, 0-8 wt. % Re, 0-1 wt. % Si, 0-1 wt. % O, and 0-1 wt. % C.

In accordance with another and/or alternative non-limiting aspect of the present disclosure, one or more portions and/or components of the formed medical device includes a zirconium oxide (ZrO₂) coating. The enhancement layer can be used to improve hardness, improve toughness, improve resistance to corrosion and oxidation, reduced friction, and/or form a reduced stick surface when in contact with many different materials. In one non-limiting embodiment all or a portion of the outer surface of the one or more portions or components of the formed medical device is initially coated with Zr metal. The Zr metal coating can be applied by PVD, CVD, ALD and PE-CVD in an inert environment. The coating thickness of Zr metal is 0.5-15 microns. Thereafter, the Zr metal coating is exposed to oxygen gas and/or oxygen containing gas compound to cause the oxygen to react with the Zn metal coating to form a layer of zirconium oxide (ZrO₂) on the outer surface of the Zr metal coating and/or the outer surface of the one or more portions or components of the formed medical device. Particles of Zr metal can optionally be mixed with oxygen gas and/or an oxygen containing gas compound to facilitate in the formation of the ZrO₂ coating. When Zr metal particles are used, the initial Zr coating layer on the outer surface of one or more portions or components of the formed medical device can optionally be eliminated. The zirconium oxide (ZrO₂) coating has been found to produce a blue colored enhancement layer color. In another and/or alternative non-limiting embodiment, the enhancement layer composition generally includes 35-90 wt. % Zr (and all values and ranges therebetween), 10-35 wt. % O (and all values and ranges therebetween), 0-2 wt. % N (and all values and ranges therebetween), 0-10 wt. % Re (and all values and ranges therebetween), 0-20 wt. % Si (and all values and ranges therebetween), and 0-2 wt. % C (and all values and ranges therebetween). In another and/or alternative non-limiting embodiment, the enhancement layer composition generally includes 70-80 wt. % Zr, 20-30 wt. %, 0-1 wt. % N, 0-8 wt. % Re, 0-1 wt. % Si, and 0-1 wt. % C.

In accordance with another and/or alternative non-limiting aspect of the present disclosure, one or more portions and/or components of the formed medical device includes both a z irconium oxide (ZrO₂) coating and a zirconium nitride coating (ZrN). The enhancement layer can be used to improve hardness, improve toughness, improve resistance to corrosion and oxidation, reduced friction, and/or form a reduced stick surface when in contact with many different materials. In one non-limiting embodiment all or a portion of the outer surface of one or more portions or components of the formed medical device is initially coated with Zr metal. The Zr metal coating can be applied by PVD, CVD, ALD and PE-CVD in an inert environment. The coating thickness of Zr metal is 0.5-15 microns. Thereafter, the Zr metal coating is exposed to a) both oxygen gas and/or oxygen containing gas compound and also to nitrogen gas and/or nitrogen containing gas compound, b) nitrogen gas and/or nitrogen containing gas compound and then to oxygen gas and/or oxygen containing gas compound, or c) oxygen gas and/or oxygen gas containing compound and then to nitrogen gas and/or nitrogen gas containing compound. The coating composition of the zirconium oxide (ZrO₂) coating and the zirconium nitride coating (ZrN) are similar or the same as discussed above. As discussed above, Particles of Zr metal can optionally be mixed with oxygen gas and/or an oxygen containing gas compound to facilitate in the formation of the ZrO₂ coating and the nitrogen gas and/or nitrogen gas containing compound to facilitate in the formation of the ZrN coating. When Zr metal particles are used, the initial Zr coating layer on the outer surface of one or more portions or components of the formed medical device can optionally be eliminated.

In accordance with another and/or alternative non-limiting aspect of the present disclosure, one or more portions and/or components of the formed medical device includes a zirconium oxycarbide (ZrOC) coating. The enhancement layer can be used to improve hardness, improve toughness, improve resistance to corrosion and oxidation, reduced friction, and/or form a reduced stick surface when in contact with many different materials. In one non-limiting embodiment all or a portion of the outer surface of one or more portions or components or the formed medical device is initially coated with Zr metal. The Zr metal coating can be applied by PVD, CVD, ALD and PE-CVD in an inert environment. The coating thickness of Zr metal is 0.5-15 microns. Thereafter, the Zr metal coating is exposed to a) both to oxygen gas and/or an oxygen containing gas compound and to carbon and/or a carbon containing gas compound (e.g., methane and/or acetylene gas), b) carbon and/or a carbon containing gas compound and then to oxygen gas and/or an oxygen containing gas compound, or c) oxygen gas and/or oxygen containing gas compound and then to carbon and/or carbon containing gas compound. Particles of Zr metal can optionally be mixed with oxygen gas and/or an oxygen containing gas compound and the carbon and/or carbon containing gas compound to facilitate in the formation of the zirconium oxycarbide (ZrOC) coating. When Zr metal particles are used, the initial Zr coating layer on the outer surface of one or more portions or components of the formed medical device can optionally be eliminated. In another and/or alternative non-limiting embodiment, the enhancement layer composition generally includes 40-95 wt. % Zr (and all values and ranges therebetween), 5-25 wt. % O (and all values and ranges therebetween), and 10-40 wt. % C (and all values and ranges therebetween), 0-2 wt. % N (and all values and ranges therebetween), 0-10 wt. % Re (and all values and ranges therebetween), and 0-20 wt. % Si (and all values and ranges therebetween). In another and/or alternative non-limiting embodiment, the enhancement layer composition generally includes 40-65 wt. % Zr, 5-25 wt. % O, and 25-40 wt. % C, 0-1 wt. % N, 0-8 wt. % Re, and 0-1 wt. % Si.

In accordance with another and/or alternative non-limiting aspect of the present disclosure, one or more portions and/or components of the formed medical device includes a zirconium oxynitride (ZrNxOy) [e.g., cubic ZrN:O, cubic ZrO₂:N, tetragonal ZrO₂:N, and monoclinic ZrO₂:N phase coatings]. A portion or all of the outer surface of the one or more portions or components of the formed medical device can include the zirconium oxynitride (ZrNxOy). The enhancement layer can be used to improve hardness, improve toughness, improve resistance to corrosion and oxidation, reduced friction, form a reduced stick surface when in contact with many different materials, and/or promote nitric oxide formation on the surface of the coating. In one non-limiting embodiment, all or a portion of the outer surface of the one or more portions or components of the formed medical device are optionally initially coated with Zr metal. The Zr metal coating, when applied, can be applied by PVD, CVD, ALD and PE-CVD in an inert environment. The coating thickness of Zr metal is 0.05-15 microns (and all values and ranges therebetween). As can be appreciated, the initial Zr coating is optional. Thereafter, the Zr metal coating is exposed to zirconium particles and a nitrogen and oxygen mixture that can include nitrogen gas, oxygen gas, a nitrogen containing gas compound and/or an oxygen containing gas compound to cause the nitrogen and oxygen to react with the Zr metal coating, if such coating is used, and/or with the Zr metal particles to form a layer of ZrNxOy on the outer surface of the Zr metal coating and/or the outer surface of the one or more portions or components of the formed medical device. The ratio of the N to the O can be varied to control the amount of O and N in the ZrNxOy coating. If a zirconium layer is not preapplied, the ZrNxOy coating can be formed by exposing the outer surface of one or more portions or components of the formed medical device to zirconium particles and a nitrogen and oxygen source such as nitrogen gas, oxygen gas, a nitrogen containing gas compound and/or an oxygen containing gas compound. The ratio of N to O when forming the ZrNxOy coating is generally 1:10 to 10:1 (and all values and ranges therebetween). The coating thickness of the ZrNxOy coating is generally at least 0.1 microns (e.g., 0.1-15 microns and all values and ranges therebetween), and typically 0.2-2 microns. In another and/or alternative non-limiting embodiment, a ZrNxOy coating is applied to a portion or all of the outer surface of the one or more portions or components of the formed medical device, and the ZrNxOy coating is formed by a) exposing the outer surface of a portion of all of the one or more portions or components of the formed medical device to Zr particles (PVD, CVD, ALD and PE-CVD process) and/or a Zr containing solution to form a Zr layer on a portion of all of the one or more portions or components of the formed medical device, and wherein the thickness of the Zr coating is 0.05-5 microns, and b) exposing the Zr coating to a nitrogen and oxygen source such as nitrogen gas, oxygen gas, a nitrogen containing gas compound and/or an oxygen containing gas compound to form a ZrNxOy coating, and wherein ratio of N to O when forming the ZrNxOy coating is generally 1:10 to 10:1, and wherein the coating thickness of the ZrNxOy coating is 0.2-5 microns. In another and/or alternative non-limiting embodiment, a ZrNxOy coating is applied to a portion or all of the outer surface of the one or more portions or components of the formed medical device, and the ZrNxOy coating is formed by exposing a portion or all of the outer surface of the one or more portions or components of the formed medical device to Zr particles and a nitrogen and oxygen source such as nitrogen gas, oxygen gas, a nitrogen containing gas compound and/or an oxygen containing gas compound to form a ZrNxOy coating, and wherein ratio of N to O when forming the ZrNxOy coating is generally 1:10 to 10:1, and wherein the coating thickness of the ZrNxOy coating is 0.2-5 microns. In another and/or alternative non-limiting embodiment, the enhancement layer composition generally includes 20-85 wt. % Zr (and all values and ranges therebetween), 0.5-35 wt. % N (and all values and ranges therebetween), and 0.5-35 wt. % O (and all values and ranges therebetween). In another and/or alternative non-limiting embodiment, a coating of ZrNxOy was formed on one or more portions or components of the formed medical device by reactive physical vapor deposition in a vacuum chamber. Depending on the oxygen-nitrogen ratio during vapor deposition, a coating deposit of ZrNxOy with defined composition and resistivity can be coated on the outer surface of the one or more portions or components of the formed medical device.

In accordance with another and/or alternative non-limiting aspect of the present disclosure, one or more portions and/or components of the formed medical device includes a zirconium-nitrogen-carbon (ZrNC) coating. The enhancement layer can be used to improve hardness, improve toughness, improve resistance to corrosion and oxidation, reduced friction, and/or form a reduced stick surface when in contact with many different materials. In one non-limiting embodiment all or a portion of the outer surface of the one or more portions or components of the formed medical device is initially coated with Zr metal. The Zr metal coating can be applied by PVD, CVD, ALD and PE-CVD in an inert environment. The coating thickness of Zr metal is 0.5-15 microns. Thereafter, the Zr metal coating is exposed to nitrogen gas and/or a nitrogen containing gas compound and then to carbon and/or a carbon containing gas compound (e.g., methane and/or acetylene gas). The color of the ZrNC will vary depending on the amount of C and N in the coating. Particles of Zr metal can optionally be mixed with nitrogen gas and/or a nitrogen containing gas compound and the carbon and/or a carbon containing gas compound to facilitate in the formation of the ZrNC coating. When Zr metal particles are used, the initial Zr coating layer on the outer surface of one or more portions or components of the formed medical device can optionally be eliminated. In one non-limiting embodiment, the enhancement layer composition generally includes 40-95 wt. % Zr (and all values and ranges therebetween), 5-40 wt. % N (and all values and ranges therebetween), and 5-40 wt. % C (and all values and ranges therebetween), 0-2 wt. % O (and all values and ranges therebetween), 0-10 wt. % Re (and all values and ranges therebetween), and 0-20 wt. % Si (and all values and ranges therebetween). In another and/or alternative non-limiting embodiment, the enhancement layer composition generally includes 40-80 wt. % Zr, 5-25 wt. % N, and 5-25 wt. % C, 0-1 wt. % O, 0-8 wt. % Re, and 0-1 wt. % Si.

In accordance with another and/or alternative non-limiting aspect of the present disclosure, a portion or all of the formed medical device is formed of a metal alloy that includes a) stainless steel, b) CoCr alloy, c) TiAlV alloy, d) aluminum alloy, e) nickel alloy, f) titanium alloy, g) tungsten alloy, h) molybdenum alloy, i) copper alloy, j) beryllium-copper alloy, k) titanium-nickel alloy, l) refractory metal alloy, or m) metal alloy (e.g., stainless steel, CoCr alloy, TiAlV

alloy, aluminum alloy, nickel alloy, titanium alloy, tungsten alloy, molybdenum alloy, copper alloy, beryllium-copper alloy, titanium-nickel alloy, refractory metal alloy, etc.) that includes at least 5 awt. % rhenium and/or hafnium, and wherein a portion or all of the outer surface of the metal alloy is coated with an enhancement layer (e.g., chromium nitride (CrN), diamond-like carbon (DLC), titanium nitride (TiN), titanium nitride oxide (TiNOx), zirconium nitride (ZrN), zirconium oxide (ZrO₂), zirconium-nitrogen-carbon (ZrNC), zirconium OxyCarbide (ZrOC), zirconium oxynitride (ZrNxOy) [e.g., cubic ZrN:O, cubic ZrO₂:N, tetragonal ZrO₂:N, and monoclinic ZrO₂:N phase coatings]), and wherein the outer surface of the metal alloy optionally includes an adhesion layer, which adhesion layer is optionally a metallic layer that includes titanium or zirconium.

In accordance with another and/or alternative non-limiting aspect of the present disclosure, a portion or all of the formed medical device is formed of a metal alloy that includes a) stainless steel, b) CoCr alloy, c) TiAlV alloy, d) aluminum alloy, e) nickel alloy, f) titanium alloy, g) tungsten alloy, h) molybdenum alloy, i) copper alloy, j) beryllium-copper alloy, k) titanium-nickel alloy, l) refractory metal alloy, or m) metal alloy (e.g., stainless steel, CoCr alloy, TiAlV alloy, aluminum alloy, nickel alloy, titanium alloy, tungsten alloy, molybdenum alloy, copper alloy, beryllium-copper alloy, titanium-nickel alloy, refractory metal alloy, etc.) that includes at least 5 awt. % rhenium and/or hafmium, and wherein the metal alloy is coated with a metal oxynitride layer (e.g., titanium nitride oxide and/or (TiNOx), zirconium oxynitride (ZrNxOy), etc.), which metal oxynitride layer can optionally be used to promotes and/or facilitates in a) formation or generation of nitric oxide (NO), b) stimulation of endothelial cells, c) a modulation of endothelial cells, d) reduce neointimal hyperplasia, e) reduce tissue proliferation, f) reduce platelet activation, g) reduce thrombosis, h) reduce restenosis, i) promote endothelial cell angiogenesis, and/or j) improved healing on and/or about the formed medical device, and wherein the outer surface of the metal alloy optionally includes an adhesion layer, which adhesion layer is optionally a metallic layer that includes titanium or zirconium, and which metal oxynitride layer is optionally partially or fully coated on the outer surface of the adhesion layer.

In accordance with another and/or alternative non-limiting aspect of the present disclosure, all or a portion of the formed medical device is formed of a titanium-nickel alloy or a titanium-nickel alloy that includes at least 5 awt. % rhenium and/or hafnium, and wherein a portion or all of the outer surface of the metal alloy is coated with a metal oxynitride layer (e.g., titanium nitride oxide and/or (TiNOx), zirconium oxynitride (ZrNxOy), etc.), and wherein all or a portion the formed medical device are optionally coated with a metal oxynitride layer, and wherein the outer surface of the metal alloy optionally includes an adhesion layer, which adhesion layer is optionally a metallic layer that includes titanium or zirconium, and which metal oxynitride layer is optionally partially or fully coated on the outer surface of the adhesion layer.

In accordance with another and/or alternative non-limiting aspect of the present disclosure, all or a portion of the medical is formed of a stainless-steel alloy or a stainless-steel alloy that includes at least 5 awt. % rhenium and/or hafnium, and wherein a portion or all of the outer surface of the metal alloy is coated with a metal oxynitride layer (e.g., titanium nitride oxide and/or (TiNOx), zirconium oxynitride (ZrNxOy), etc.), and wherein all or a portion of the formed medical device is optionally coated with a metal oxynitride layer, and wherein the outer surface of the metal alloy optionally includes an adhesion layer, which adhesion layer is optionally a metallic layer that includes titanium or zirconium, and which metal oxynitride layer is optionally partially or fully coated on the outer surface of the adhesion layer.

In accordance with another and/or alternative non-limiting aspect of the present disclosure, all or a portion of the formed medical device is formed of a cobalt-chromium alloy or a cobalt-chromium alloy that includes at least 5 awt. % rhenium and/or hafnium, and wherein a portion or all of the outer surface of the metal alloy is coated with a metal oxynitride layer (e.g., titanium nitride oxide and/or (TiNOx), zirconium oxynitride (ZrNxOy), etc.), and wherein all or a portion of components of the formed medical device is optionally coated with a metal oxynitride layer, and wherein the outer surface of the metal alloy optionally includes an adhesion layer, which adhesion layer is optionally a metallic layer that includes titanium or zirconium, and which metal oxynitride layer is optionally partially or fully coated on the outer surface of the adhesion layer.

In accordance with another and/or alternative non-limiting aspect of the present disclosure, all or a portion of the formed medical device is formed of a TiAlV alloy or a TiAlV alloy that includes at least 5 awt. % rhenium and/or hafnium, and wherein a portion or all of the outer surface of the metal alloy is coated with a metal oxynitride layer (e.g., titanium nitride oxide and/or (TiNOx), zirconium oxynitride (ZrNxOy), etc.), and wherein all or a portion of the formed medical device is optionally coated with a metal oxynitride layer, and wherein the outer surface of the metal alloy optionally includes an adhesion layer, which adhesion layer is optionally a metallic layer that includes titanium or zirconium, and which metal oxynitride layer is optionally partially or fully coated on the outer surface of the adhesion layer.

In accordance with another and/or alternative non-limiting aspect of the present disclosure, all or a portion of the formed medical device is formed of a refractory metal alloy or a refractory metal alloy that includes at least 5 awt. % rhenium and/or hafnium, and wherein a portion or all of the outer surface of the metal alloy is coated with a metal oxynitride layer (e.g., titanium nitride oxide and/or (TiNOx), zirconium oxynitride (ZrNxOy), etc.), and wherein all or a portion of the formed medical device is optionally coated with a metal oxynitride layer, and wherein the outer surface of the metal alloy optionally includes an adhesion layer, which adhesion layer is optionally a metallic layer that includes titanium or zirconium, and which metal oxynitride layer is optionally partially or fully coated on the outer surface of the adhesion layer.

In accordance with another and/or alternative non-limiting aspect of the present disclosure, all or a portion of the formed medical device is formed of a metal alloy that includes at least 5 awt. % rhenium and/or hafnium, and wherein a portion or all of the outer surface of the metal alloy is coated with a metal oxynitride layer (e.g., titanium nitride oxide and/or (TiNOx), zirconium oxynitride (ZrNxOy), etc.), and wherein all or a portion of the formed medical device optionally coated with a metal oxynitride layer, and wherein the outer surface of the formed medical device that includes the metal oxynitride layer optionally includes an adhesion layer, which adhesion layer is optionally a metallic layer that includes titanium or zirconium, and which metal oxynitride layer is optionally partially or fully coated on the outer surface of the adhesion layer.

In accordance with another and/or alternative non-limiting aspect of the present disclosure, there is provided a method for forming a medical device that is partially or fully formed of a metal alloy that includes at least 5 atomic percent of hafnium and/or rhenium comprising a) providing rhenium and/or hafnium powder and one or more metal powder additives selected from the group of molybdenum, calcium, chromium, cobalt, copper, gold, iron, magnesium, nickel, niobium, osmium, platinum, rare earth metals, rhenium, silver, tantalum, technetium, titanium, tungsten, vanadium, yttrium, zinc, and/or zirconium, and/or an powder alloy of such metals, and wherein the powder particles are less than 1000 microns in size, b) consolidating the metal powder under pressure to form a green part or component, and wherein the step of consolidation optionally occurs at a pressure of 400-700 MPa, c) sintering the green part or component, and wherein the step of sintering optionally occurs at 1000-3000° C. to fuse the metal powders together, and wherein the step of sintering is optionally performed in a reduced oxygen atmosphere (e.g., argon environment, non-oxygen containing environment, inert environment, etc.) and/or under a vacuum, and wherein the sintered green part or component has an as-sintered average density of about 90-99% of a minimum theoretical density of a metal alloy formed from melting the metal powder, d) optionally subjecting the green part or component to a swaging operation to form a metal plate, and wherein the swaging process reduces the cross-sectional area of the metal plate, and wherein the swaging process optionally occurs at a temperature of 400° C.-1500° C. in a controlled neutral or non-reducing environment, e) optionally annealing the metal plate, and wherein the optional annealing occurs in a reduced oxygen atmosphere (e.g., argon environment, non-oxygen containing environment, inert environment, etc.) and/or under a vacuum, f) cutting the metal plate to partially or fully form one or more medical devices from the metal plate, g) optionally separating the one or more medical devices from the cut metal plate, h) optionally smoothing and polishing the one or more medical devices, i) bending one or more of the medical devices into its final or near final shape, k) optionally providing a coating material; said coating material is formed of a different material from said metal alloy; and l) optionally coating the coating material on at least a portion of an outer surface of one or more of the medical devices.

One non-limiting object of the present disclosure is the provision of a method and process for manufacturing medical devices in a timely and cost-effective manner, while also maintaining the stringent dimension parameters of such medical devices.

In another and/or alternative non-limiting object of the present disclosure is the provision of a method and process for manufacturing medical devices (e.g., bone plates and other orthopedic medical devices) from flat metal plates.

In another and/or alternative non-limiting object of the present disclosure is the provision of a method and process for manufacturing medical devices that includes the steps of a) optionally forming a flat planar metal plate or providing a stock metal plate, and wherein the flat planar metal plate optionally has 0-2° curvature (and all values and ranges therebetween), and wherein the cross-sectional area of the metal plate and the thickness of the metal plate are non-limiting, and wherein the thickness of the metal plate is constant over 80-100% (and all values and ranges therebetween) of the cross-sectional area of the metal plate, b) cutting and/or machining the metal plate using one or more processes (e.g., water jet cutting, laser cutting, EDM cutting, CNC machining, electrochemical etching, grinding, shearing, polishing, stamping, etc.) to form one or more medical devices from the metal plate, c) optionally separating the one or more medical devices from the metal plate (e.g., cutting the one or more medical devices from the metal plate that was not used to form the one or more medical devices, shearing the one or more medical devices from the metal plate that was not used to form the one or more medical devices, grinding off one or more medical devices from the metal plate that was not used to form the one or more medical devices, etc.), d) optionally smoothing one or more of the edges (e.g., smoothing the outer perimeter edges of the medical device, smoothing the edges of one or more openings that are spaced from the perimeter edge of the medical device, etc.) by use of one or more processes (e.g., polishing, grinding, etc.), e) optionally grinding down and smoothing the one or more regions of the medical device that needed to be cut or separated by some other method when the medical device was separated from the metal plate, f) bending (e.g., bending by use of a hydraulic press, bending by use of mating dies, etc.) the one or more medical devices to form a near final or final shape of the one or more medical devices, g) optionally further cutting (e.g., water jet cutting, laser cutting, EDM cutting, CNC machining, drilling, gun drilling, electrochemical etching, shearing, grinding, etc.) the medical device to form the final form and shape of the medical device, and wherein this optional cutting process can occur prior to and/or after the step of bending, h) optionally applying a coating to a portion or all of the outer surface of the medical device prior to and/or after the bending of the medical device into its final shape, and wherein the coating can be one or more of A) a metal coating that has a composition that is different from the composition of the metal plate, B) a biological agent coating, C) a nitride coating, D) an enhancement coating, and/or E) a ceramic coating other than an enhancement coating, and wherein the optional coating can be applied prior to or after the step of bending, the step of cutting, and/or the step of grinding and/or smoothing.

In another and/or alternative non-limiting object of the present disclosure is the provision of a method and process for manufacturing medical devices and a) the flat planar metal plate (e.g., plate has 0-2° curvature and all values and ranges therebetween), b) the top and/or bottom surface of the metal plate can optionally be polished to have a smooth top and/or bottom surface, c) the shape of the metal plate is optionally square or rectangular; however, other shapes can be used, d) he thickness of the metal plate can be constant over 80-100% (and all values and ranges therebetween) of the cross-sectional area of the metal plate, and wherein the thickness of the metal plate is optionally 0.01 mm to 10 mm (and all values and ranges therebetween), and/or e) prior to the metal plate being cut, the plate can be optionally covered and/or sealed in a material (e.g., plastic, etc.) to inhibit or prevent oxidation of the surface of the plate, and wherein prior to the further processing of the plate (e.g., cutting, bending, coating, etc.) the material used for sealing, when used, is typically removed.

In another and/or alternative non-limiting object of the present disclosure is the provision of a method and process for manufacturing medical devices wherein the metal plate is partially (e.g. 1-99.999 wt. % and all values and ranges therebetween) or fully formed of a metal material that includes a) stainless steel, b) CoCr alloy, c) TiAlV alloy, d) aluminum alloy, e) nickel alloy, f) titanium alloy, g) tungsten alloy, h) molybdenum alloy, i) copper alloy, j) beryllium-copper alloy, k) titanium-nickel alloy, l) refractory metal alloy, m) metal alloy (e.g., stainless steel, CoCr alloy, TiAlV alloy, aluminum alloy, nickel alloy, titanium alloy, tungsten alloy, molybdenum alloy, copper alloy, beryllium-copper alloy, titanium-nickel alloy, refractory metal alloy, etc.) that is modified to further include at least 5 atomic weight percent (awt. %) or atomic percent (awt. %) rhenium (e.g., 5-99 awt. % rhenium and all values and ranges therebetween), n) metal alloy (e.g.,

stainless steel, CoCr alloy, TiAlV alloy, aluminum alloy, nickel alloy, titanium alloy, tungsten alloy, molybdenum alloy, copper alloy, beryllium-copper alloy, titanium-nickel alloy, refractory metal alloy, etc.) that is modified to further include at least 5 atomic weight percent (awt. %) or atomic percent (awt. %) hafnium (e.g., 5-99 awt. % hafnium and all values and ranges therebetween), o) at least 5 awt. % (e.g., 5-99 awt. % and all values and ranges therebetween) hafnium, and 0.1-96 wt. % (and all values and ranges therebetween) of one or more additives selected from the group of aluminum, boron, beryllium, bismuth, cadmium, calcium, cerium, chromium, cobalt, copper, gallium, gold, iridium, iron, lanthanum, lithium, magnesium, manganese, molybdenum, nickel, niobium, osmium, palladium, platinum, rare earth metals, rhenium, rhodium, ruthenium, scandium, silver, silicon, tantalum, technetium, tin, titanium, tungsten, vanadium, yttrium, zinc, and/or zirconium, and the metal alloy optionally includes 0-2 wt. % (and all values and ranges therebetween) of a combination of other components other than the additives (e.g., carbon, oxygen, phosphorous, sulfur, hydrogen, lead, nitrogen, etc.), or p) at least 5 awt. % (e.g., 5-99 awt. % and all values and ranges therebetween) rhenium, and 0.1-96 wt. % (and all values and ranges therebetween) of one or more additives selected from the group of aluminum, boron, beryllium, bismuth, cadmium, calcium, cerium, chromium, cobalt, copper, gallium, gold, hafnium, iridium, iron, lanthanum, lithium, magnesium, manganese, molybdenum, nickel, niobium, osmium, palladium, platinum, rare earth metals, rhodium, ruthenium, scandium, silver, silicon, tantalum, technetium, tin, titanium, tungsten, vanadium, yttrium, zinc, and/or zirconium, and the metal alloy optionally includes 0-2 wt. % (and all values and ranges therebetween) of a combination of other components other than the additives (e.g., carbon, oxygen, phosphorous, sulfur, hydrogen, lead, nitrogen, etc.).

In another and/or alternative non-limiting object of the present disclosure is the provision of a method and process for manufacturing medical devices wherein the metal plate is formed of a metal alloy that has a reduced ion release when the medical device is implanted in a patient, and wherein the metal alloy optionally has a maximum ion release of the primary metal alloy components into the tissue and/or fluid surrounding metal alloy that has been implanted into a patient of no more than 0.5 μg/cm² per day (e.g., 0-0.5 μg/cm² per day and all values and ranges therebetween).

In another and/or alternative non-limiting object of the present disclosure is the provision of a method and process for manufacturing medical devices wherein the metal plate is formed of a metal alloy that has a hydrophilicity wherein the contact angle of a water droplet on the surface of the metal alloy is 25°-45° (and all values and ranges therebetween), and typically 30-42°.

In another and/or alternative non-limiting object of the present disclosure is the provision of a method and process for manufacturing medical devices wherein the metal plate is formed from metal powder that has been consolidated to form of a green plate that has the same or similar shape and size as the plate to be used to form the medical device, and wherein the pressing process is by an isostatic process (i.e., uniform pressure applied from all sides on the metal powder); however other processes can be used, and wherein pressing process can be performed in an inert atmosphere, an oxygen-reducing atmosphere (e.g., hydrogen, argon and hydrogen mixture, etc.), and/or under a vacuum; however, this is not required, and wherein the average particle size of the metal powders used to form the metal plate is less than about 230 mesh (e.g., less than 63 microns; 1-62 microns and all values and ranges therebetween), and wherein the average density of the metal powders used to form the metal plate is optionally greater than 5 g/cm3 (e.g., 5.001 g/cm3 to 19.3 g/cm3 and all values and ranges therebetween). and wherein 10-100 vol. % (and all values and ranges therebetween) of the metal powder that is used to form the metal plate is optionally spherical shaped, and wherein the metal powder used to form the metal alloy optionally has a purity grade of at least 99.9 and more typically at least about 99.95, and wherein the average density of the near net metal that is obtained by pressing together the metal powders is about 80-95% (and all values and ranges therebetween) of the final average density of the metal plate, and wherein pressing pressures are optionally at least about 300 MPa are generally used, and wherein after the metal powders are pressed together, the pressed metal powders are optionally sintered to partially or fully fuse the metal powders together to form the green part or green metal plate, and wherein the sintering of the consolidated metal powder can optionally be performed in an oxygen-reduced atmosphere (e.g., helium, argon, hydrogen, argon and hydrogen mixture, etc.), and/or under a vacuum, and/or some other non-oxidizing environment, and wherein the sintered metal powder optionally has an as-sintered average density of about 90-99% the minimum theoretical density of the metal alloy that is used to form the metal plate, and wherein the sintering optionally occurs at 1000-3000° C. to fuse the metal powders together, and wherein the sintering is optionally performed in a reduced oxygen atmosphere (e.g., argon environment, non-oxygen containing environment, inert environment, etc.) and/or under a vacuum.

In another and/or alternative non-limiting object of the present disclosure is the provision of a method and process for manufacturing medical devices wherein the metal plate is optionally treated by a swaging process, and wherein swaging temperatures can be from 10° C. to 1500° C. and all values and ranges therebetween, and wherein the swaging can optionally be conducted in air or an oxidizing environment, or a controlled neutral or non-reducing environment (e.g., inert environment, non-oxidizing environment, vacuum, etc.), and wherein during the swaging process ions of boron and/or nitrogen are optionally allowed to impinge upon rhenium and/or hafnium atoms in the metal alloy that include rhenium and/or hafnium so as to form ReB₂, ReN₂, ReN₃, HfB₂, HfN₂ and/or HfN₃.

In another and/or alternative non-limiting object of the present disclosure is the provision of a method and process for manufacturing medical devices wherein the metal plate is optionally annealed to soften the metal plate and then machined and/or cut into a desired shape, wherein after the metal plate has been optionally machined, cut and/or shaped, the metal plate can optionally be re-hardened (e.g., subjecting the metal plate to a swaging process, etc.).

In another and/or alternative non-limiting object of the present disclosure is the provision of a method and process for manufacturing medical devices wherein the metal plate is optionally be nitrided.

In another and/or alternative non-limiting object of the present disclosure is the provision of a method and process for manufacturing medical devices wherein the metal plate is cut and/or machined using one or more processes (e.g., water jet cutting, CNC machining, electrochemical etching, grinding, gun drilling, shearing, etc.) to partially (e.g., form 60-99.99% of one or more of the medical devices from the metal plate and all values and ranges therebetween) or fully form one or more medical devices from the metal plate.

In another and/or alternative non-limiting object of the present disclosure is the provision of a method and process for manufacturing medical devices wherein the cutting process leaves one or more connection points between the one or more medical devices and the plate so that the one or more cut medical devices remain connected to the metal plate; however, this is not required.

In another and/or alternative non-limiting object of the present disclosure is the provision of a method and process for manufacturing medical devices wherein other or additional process can be used to smooth and/or round-off the edges (e.g., polishing, grinding, articulating water jet, etc.).

In another and/or alternative non-limiting object of the present disclosure is the provision of a method and process for manufacturing medical devices wherein after the formed medical device is separated from the metal plate, one or more regions of the formed medical device can optionally be bent to form the final shape of the medical device, and wherein the bending can be accomplished by one or more processes (e.g., bending by use of a hydraulic press, bending by use of mating dies, etc.).

In another and/or alternative non-limiting object of the present disclosure is the provision of a method and process for manufacturing medical devices wherein the formed medical device can include, contain, and/or be coated with one or more agents that facilitate in the success of the formed medical device and/or treated area, and wherein the “agent” includes, but is not limited to a substance, pharmaceutical, biologic, veterinary product, drug, and analogs or derivatives otherwise formulated and/or designed to prevent, inhibit and/or treat one or more clinical and/or biological events, and/or to promote healing, and wherein the agent is a material other than an enhancement coating.

In another and/or alternative non-limiting object of the present disclosure is the provision of a method and process for manufacturing medical devices wherein the formed medical device can be partially (e.g., 1% to 99.99% and all values and ranges therebetween) or fully be coated with an enhancement layer to improve one or more properties of the formed medical device (e.g., change exterior color of material having coated surface, increase surface hardness by use of the coated surface, increase surface toughness material having coated surface, reduced friction via use of the coated surface, improve scratch resistance of material that has the coated surface, improve impact wear of coated surface, improve resistance to corrosion and oxidation of coated material, form a non-stick coated surface, improve biocompatibility of material having the coated surface, reduce toxicity of material having the coated surface, reduce ion release from material having the coated surface, the enhancement layer forms a surface that is less of an irritant to cell about the coated surface after the formed medical device is implanted, reduces the rate to which cells grown on coated surface after the formed medical device is implanted, reduce rate to which movable components in the formed medical device fail to properly operate after orthopedic device is implanted, etc.), and wherein the enhancement layer is optionally includes chromium nitride (CrN), diamond-like carbon (DLC), titanium nitride (TiN), titanium oxynitride or titanium nitride oxide (TiNOx), zirconium nitride (ZrN), zirconium oxide (ZrO₂), zirconium oxynitride (ZrNxOy) [e.g., cubic ZrN:O, cubic ZrO₂:N, tetragonal ZrO₂:N, and monoclinic ZrO₂:N phase coatings], oxyzirconium-nitrogen-carbon (ZrNC), zirconium OxyCarbide (ZrOC), and combinations of such coatings, and wherein the one or more enhancement layers are optionally applied to a portion or all of the outer surface of the formed medical device by a vacuum process using an energy source to vaporize material and deposit a thin layer of enhancement layer material, and wherein the vacuum coating process, when used, can include 1a physical vapor deposition (PVD) process (e.g., sputter deposition, cathodic arc deposition or electron beam heating, etc.), chemical vapor deposition (CVD) process, atomic layer deposition (ALD) process, or a plasma-enhanced chemical vapor deposition (PE-CVD) process.

In another and/or alternative non-limiting object of the present disclosure is the provision of a method and process for manufacturing medical devices wherein a portion or all of the formed medical device is formed of a metal alloy that includes a) stainless steel, b) CoCr alloy, c) TiAlV alloy, d) aluminum alloy, e) nickel alloy, f) titanium alloy, g) tungsten alloy, h) molybdenum alloy, i) copper alloy, j) beryllium-copper alloy, k) titanium-nickel alloy, l) refractory metal alloy, or m) metal alloy (e.g., stainless steel, CoCr alloy, TiAlV alloy, aluminum alloy, nickel alloy, titanium alloy, tungsten alloy, molybdenum alloy, copper alloy, beryllium-copper alloy, titanium-nickel alloy, refractory metal alloy, etc.) that includes at least 5 awt. % rhenium and/or hafnium, and wherein the metal alloy is coated with a metal oxynitride layer (e.g., titanium nitride oxide and/or (TiNOx), zirconium oxynitride (ZrNxOy), etc.), which metal oxynitride layer can optionally be used to promotes and/or facilitates in a) formation or generation of nitric oxide (NO), b) stimulation of endothelial cells, c) a modulation of endothelial cells, d) reduce neointimal hyperplasia, e) reduce tissue proliferation, f) reduce platelet activation, g) reduce thrombosis, h) reduce restenosis, i) promote endothelial cell angiogenesis, and/or j) improved healing on and/or about the formed medical device, and wherein the outer surface of the metal alloy optionally includes an adhesion layer, which adhesion layer is optionally a metallic layer that includes titanium or zirconium, and which metal oxynitride layer is optionally partially or fully coated on the outer surface of the adhesion layer.

In another and/or alternative non-limiting object of the present disclosure is the provision of a method and process for manufacturing medical devices wherein a portion or all of the formed medical device is formed of a metal alloy that includes a titanium-nickel alloy or a titanium-nickel alloy that includes at least 5 awt. % rhenium and/or hafnium, and wherein a portion or all of the outer surface of the metal alloy is coated with a metal oxynitride layer (e.g., titanium nitride oxide and/or (TiNOx), zirconium oxynitride (ZrNxOy), etc.), and wherein all or a portion the formed medical device are optionally coated with a metal oxynitride layer, and wherein the outer surface of the metal alloy optionally includes an adhesion layer, which adhesion layer is optionally a metallic layer that includes titanium or zirconium, and which metal oxynitride layer is optionally partially or fully coated on the outer surface of the adhesion layer.

In another and/or alternative non-limiting object of the present disclosure is the provision of a method and process for manufacturing medical devices wherein a portion or all of the formed medical device is formed of a metal alloy that includes a stainless-steel alloy or a stainless-steel alloy that includes at least 5 awt. % rhenium and/or hafnium, and wherein a portion or all of the outer surface of the metal alloy is coated with a metal oxynitride layer (e.g., titanium nitride oxide and/or (TiNOx), zirconium oxynitride (ZrNxOy), etc.), and wherein all or a portion of the formed medical device is optionally coated with a metal oxynitride layer, and wherein the outer surface of the metal alloy optionally includes an adhesion layer, which adhesion layer is optionally a metallic layer that includes titanium or zirconium, and which metal oxynitride layer is optionally partially or fully coated on the outer surface of the adhesion layer.

In another and/or alternative non-limiting object of the present disclosure is the provision of a method and process for manufacturing medical devices wherein a portion or all of the formed medical device is formed of a metal alloy that includes a cobalt-chromium alloy or a cobalt-chromium alloy that includes at least 5 awt. % rhenium and/or hafnium, and wherein a portion or all of the outer surface of the metal alloy is coated with a metal oxynitride layer (e.g., titanium nitride oxide and/or (TiNOx), zirconium oxynitride (ZrNxOy), etc.), and wherein all or a portion of components of the formed medical device is optionally coated with a metal oxynitride layer, and wherein the outer surface of the metal alloy optionally includes an adhesion layer, which adhesion layer is optionally a metallic layer that includes titanium or zirconium, and which metal oxynitride layer is optionally partially or fully coated on the outer surface of the adhesion layer.

In another and/or alternative non-limiting object of the present disclosure is the provision of a method and process for manufacturing medical devices wherein a portion or all of the formed medical device is formed of a metal alloy that includes a TiAlV alloy or a TiAlV alloy that includes at least 5 awt. % rhenium and/or hafnium, and wherein a portion or all of the outer surface of the metal alloy is coated with a metal oxynitride layer (e.g., titanium nitride oxide and/or (TiNOx), zirconium oxynitride (ZrNxOy), etc.), and wherein all or a portion of the formed medical device is optionally coated with a metal oxynitride layer, and wherein the outer surface of the metal alloy optionally includes an adhesion layer, which adhesion layer is optionally a metallic layer that includes titanium or zirconium, and which metal oxynitride layer is optionally partially or fully coated on the outer surface of the adhesion layer.

In another and/or alternative non-limiting object of the present disclosure is the provision of a method and process for manufacturing medical devices wherein a portion or all of the formed medical device is formed of a metal alloy that includes a refractory metal alloy or a refractory metal alloy that includes at least 5 awt. % rhenium and/or hafnium, and wherein a portion or all of the outer surface of the metal alloy is coated with a metal oxynitride layer (e.g., titanium nitride oxide and/or (TiNOx), zirconium oxynitride (ZrNxOy), etc.), and wherein all or a portion of the formed medical device is optionally coated with a metal oxynitride layer, and wherein the outer surface of the metal alloy optionally includes an adhesion layer, which adhesion layer is optionally a metallic layer that includes titanium or zirconium, and which metal oxynitride layer is optionally partially or fully coated on the outer surface of the adhesion layer.

In another and/or alternative non-limiting object of the present disclosure is the provision of a method and process for manufacturing medical devices wherein a portion or all of the formed medical device is formed of a metal alloy that includes a metal alloy that includes at least 5 awt. % rhenium and/or hafnium, and wherein a portion or all of the outer surface of the metal alloy is coated with a metal oxynitride layer (e.g., titanium nitride oxide and/or (TiNOx), zirconium oxynitride (ZrNxOy), etc.), and wherein all or a portion of the formed medical device optionally coated with a metal oxynitride layer, and wherein the outer surface of the formed medical device that includes the metal oxynitride layer optionally includes an adhesion layer, which adhesion layer is optionally a metallic layer that includes titanium or zirconium, and which metal oxynitride layer is optionally partially or fully coated on the outer surface of the adhesion layer.

In another and/or alternative non-limiting object of the present disclosure is the provision of a method and process for manufacturing medical devices wherein a portion or all of the formed medical device is formed of a metal alloy that includes at least 5 atomic percent of hafnium and/or rhenium comprising a) providing rhenium and/or hafnium powder and one or more metal powder additives selected from the group of molybdenum, calcium, chromium, cobalt, copper, gold, iron, magnesium, nickel, niobium, osmium, platinum, rare earth metals, rhenium, silver, tantalum, technetium, titanium, tungsten, vanadium, yttrium, zinc, and/or zirconium, and/or an powder alloy of such metals, and wherein the powder particles are less than 1000 microns in size, b) consolidating the metal powder under pressure to form a green part or component, and wherein the step of consolidation optionally occurs at a pressure of 400-700 MPa, c) sintering the green part or component, and wherein the step of sintering optionally occurs at 1000-3000° C. to fuse the metal powders together, and wherein the step of sintering is optionally performed in a reduced oxygen atmosphere (e.g., argon environment, non-oxygen containing environment, inert environment, etc.) and/or under a vacuum, and wherein the sintered green part or component has an as-sintered average density of about 90-99% of a minimum theoretical density of a metal alloy formed from melting the metal powder, d) optionally subjecting the green part or component to a swaging operation to form a metal plate, and wherein the swaging process reduces the cross-sectional area of the metal plate, and wherein the swaging process optionally occurs at a temperature of 400° C.-1500° C. in a controlled neutral or non-reducing environment, e) optionally annealing the metal plate, and wherein the optional annealing occurs in a reduced oxygen atmosphere (e.g., argon environment, non-oxygen containing environment, inert environment, etc.) and/or under a vacuum, f) cutting the metal plate to partially or fully form one or more medical devices from the metal plate, g) optionally separating the one or more medical devices from the cut metal plate, h) optionally smoothing and polishing the one or more medical devices, i) bending one or more of the medical devices into its final or near final shape, k) optionally providing a coating material; said coating material is formed of a different material from said metal alloy; and l) optionally coating the coating material on at least a portion of an outer surface of one or more of the medical devices.

In another and/or alternative non-limiting object of the present disclosure is the provision of a method and process for manufacturing medical devices wherein a portion or all of the formed medical device is formed of a metal alloy that includes the steps of a) providing a flat planar metal plate (e.g., plate has 0-2° curvature and all values and ranges therebetween), and wherein the cross-sectional area of the metal plate and the thickness of the metal plate are non-limiting, and wherein the thickness of the metal plate is constant over 80-100% (and all values and ranges therebetween) of the cross-sectional area of the metal plate, and wherein the step of providing a flat planar metal plate optionally includes the steps of forming the flat planar metal plate from metal powder, b) cutting and/or machining the metal plate using one or more processes (e.g., water jet cutting, CNC machining, electrochemical etching, grinding, etc.) to form one or more medical devices from the metal plate, c) optionally separating the one or more medical devices from the metal plate (e.g., cutting the one or more medical devices from the metal plate that was not used to form the one or more medical devices, shearing the one or more medical devices from the metal plate that was not used to form the one or more medical devices, grinding off one or more medical devices from the metal plate that was not used to form the one or more medical devices, etc.), d) optionally smoothing the edges (e.g., smoothing the outer perimeter edges of the medical device, smoothing the edges of one or more openings that are spaced from the perimeter edge of the medical device, etc.) by use of one or more processes (e.g., polishing, grinding, etc.), e) optionally grinding down and smoothing the one or more regions of the medical device that needed to be cut or separated by some other method when the medical device was separated from the metal plate, f) bending (e.g., bending by use of a hydraulic press, bending by use of mating dies, etc.) the one or more medical devices to form a final shape of the one or more medical devices, g) optionally annealing the metal plate, and h) and optionally coating a portion or all of one or more medical devices.

These and other objects and advantages will become apparent to those skilled in the art upon reading and following the description taken together with the accompanying drawings.

### BRIEF DESCRIPTION OF THE DRAWINGS

Non-limiting and non-exhaustive embodiments are described with reference to the following drawings, wherein like labels refer to like parts throughout the various views unless otherwise specified. The sizes and relative positions of elements in the drawings are not necessarily drawn to scale. For example, the shapes of various elements are selected, enlarged, and positioned to improve drawing legibility. The particular shapes of the elements as drawn have been selected for ease of recognition in the drawings. Reference may now be made to the drawings, which illustrate various embodiments that the disclosure may take in physical form and in certain parts and arrangement of parts wherein:

FIG. 1 illustrates a non-limiting metal plate 100 that can be used to form one or more medical devices. The metal plate is illustrated as being optionally covered with a protective plastic cover or sleeve that can be used to inhibit or prevent oxidation of the outer surface of the metal plate.

FIG. 2 illustrates a plurality of medical devices formed in the metal plate 100. As illustrated in FIG. 2, different shaped and sized medical devices 200 can optionally be formed in the metal plate such as one or more slot, holes 220 and the like; however, it can be appreciated that the metal plate can includes only the same shaped and sized medical devices. The medical devices 200 includes connection points 230 and an outer slot perimeter 220 that defines the outer perimeter 210 of each of the medical devices 200.

### DESCRIPTION OF NON-LIMITING EMBODIMENTS

Although specific terms are used in the following description for the sake of clarity, these terms are intended to refer only to the particular structure of the embodiments selected for illustration in the drawings and are not intended to define or limit the scope of the disclosure. In the drawings and the following description below, it is to be understood that like numeric designations refer to components of like function.

The singular forms “a,” “an,” and “the” include plural referents unless the context clearly dictates otherwise.

As used in the specification and in the claims, the term “comprising” may include the embodiments “consisting of” and “consisting essentially of.” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated ingredients/steps, which allows the presence of only the named ingredients/steps, along with any unavoidable impurities that might result therefrom, and excludes other ingredients/steps.

Numerical values in the specification and claims of this application should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently combinable (for example, the range of “from 2 grams to 10 grams” is inclusive of the endpoints, 2 grams and 10 grams, and all the intermediate values).

The terms “about” and “approximately” can be used to include any numerical value that can vary without changing the basic function of that value. When used with a range, “about” and “approximately” also disclose the range defined by the absolute values of the two endpoints, e.g. “about 2 to about 4” also discloses the range “from 2 to 4.” Generally, the terms “about” and “approximately” may refer to plus or minus 10% of the indicated number.

Percentages of elements should be assumed to be percent by weight of the stated element, unless expressly stated otherwise.

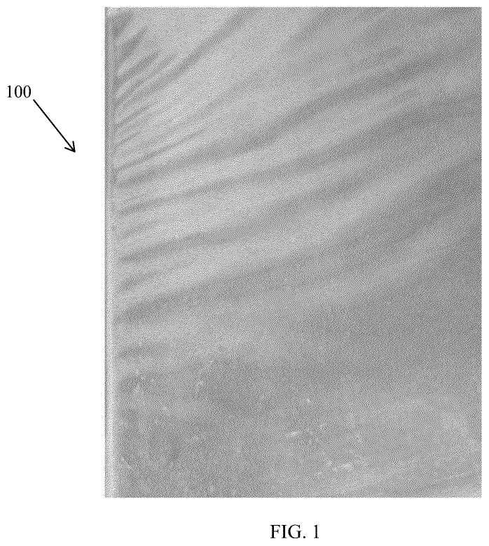

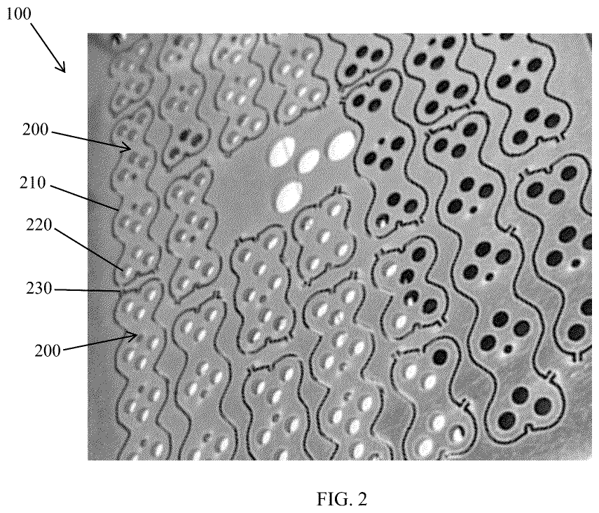

FIGS. 1-2 illustrate a medical device that can be formed from a metal plate in and accordance with the present disclosure.

In another and/or alternative non-limiting object of the present disclosure is the provision of a method and process for manufacturing medical devices wherein a portion or all of the formed medical device is formed of a metal alloy that includes at least 5 atomic percent of hafnium and/or rhenium comprising a) providing rhenium and/or hafnium powder and one or more metal powder additives selected from the group of molybdenum, calcium, chromium, cobalt, copper, gold, iron, magnesium, nickel, niobium, osmium, platinum, rare earth metals, rhenium, silver, tantalum, technetium, titanium, tungsten, vanadium, yttrium, zinc, and/or zirconium, and/or an powder alloy of such metals, and wherein the powder particles are less than 1000 microns in size, b) consolidating the metal powder under pressure to form a green part or component, and wherein the step of consolidation optionally occurs at a pressure of 400-700 MPa, c) sintering the green part or component, and wherein the step of sintering optionally occurs at 1000-3000° C. to fuse the metal powders together, and wherein the step of sintering is optionally performed in a reduced oxygen atmosphere (e.g., argon environment, non-oxygen containing environment, inert environment, etc.) and/or under a vacuum, and wherein the sintered green part or component has an as-sintered average density of about 90-99% of a minimum theoretical density of a metal alloy formed from melting the metal powder, d) optionally subjecting the green part or component to a swaging operation to form a metal plate, and wherein the swaging process reduces the cross-sectional area of the metal plate, and wherein the swaging process optionally occurs at a temperature of 400° C.-1500° C. in a controlled neutral or non-reducing environment, e) optionally annealing the metal plate, and wherein the optional annealing occurs in a reduced oxygen atmosphere (e.g., argon environment, non-oxygen containing environment, inert environment, etc.) and/or under a vacuum, f) cutting the metal plate to partially or fully form one or more medical devices from the metal plate, g) optionally separating the one or more medical devices from the cut metal plate, h) optionally smoothing and polishing the one or more medical devices, i) bending one or more of the medical devices into its final or near final shape, k) optionally providing a coating material; said coating material is formed of a different material from said metal alloy; and l, optionally coating the coating material on at least a portion of an outer surface of one or more of the medical devices.

It will thus be seen that the objects set forth above, among those made apparent from the preceding description, are efficiently attained, and since certain changes may be made in the constructions set forth without departing from the spirit and scope of the disclosure, it is intended that all matter contained in the above description and shown in the accompanying drawings shall be interpreted as illustrative and not in a limiting sense. The disclosure has been described with reference to preferred and alternate embodiments. Modifications and alterations will become apparent to those skilled in the art upon reading and understanding the detailed discussion of the disclosure provided herein. This disclosure is intended to include all such modifications and alterations insofar as they come within the scope of the present disclosure. It is also to be understood that the following claims are intended to cover all of the generic and specific features of the disclosure herein described and all statements of the scope of the disclosure, which, as a matter of language, might be said to fall therebetween.

To aid the Office and any readers of this application and any resulting patent in interpreting the claims appended hereto, Applicant does not intend any of the appended claims or claim elements to invoke 35 U.S.C. 112(f) unless the words “means for” or “step for” are explicitly used in the particular claim.

What is claimed:

1. A method and process for manufacturing medical devices wherein a portion or all of the formed medical device is formed of a metal alloy that includes at least 5 atomic percent of hafnium and/or rhenium; said method comprises:
a. providing rhenium and/or hafnium powder and one or more metal powder additives selected from the group of molybdenum, calcium, chromium, cobalt, copper, gold, iron, magnesium, nickel, niobium, osmium, platinum, rare earth metals, rhenium, silver, tantalum, technetium, titanium, tungsten, vanadium, yttrium, zinc, and/or zirconium, and/or a powder alloy of such metals; said metal powder particles are less than 1000 microns in size;
b. consolidating said metal powder under pressure to form a green part or component; said step of consolidating occurs at a pressure of at least 400 Pa;
c. sintering said green part or component; said step of sintering occurs at least 500° C. to at least partially fuse said metal powders together; said step of sintering is performed in a reduced oxygen atmosphere and/or under a vacuum; said sintered green part or component has an as-sintered average density of about 90-99% of a minimum theoretical density of a metal alloy formed from melting said metal powder;
d. subjecting the green part or component to a swaging operation to form a metal plate; said swaging process reduces a cross-sectional area of said metal plate; said swaging process occurs at a temperature of at least 20° C. in a controlled neutral or non-reducing environment;
e. annealing said metal plate; said step of annealing occurs in a reduced oxygen atmosphere and/or under a vacuum;
f. cutting the metal plate to partially or fully form one or more medical devices from the metal plate;
g. separating said one or more medical devices from said cut metal plate;
h. smoothing and polishing said one or more medical devices;
i. bending one or more of said medical devices into its final or near final shape;
j. providing a coating material; said coating material is formed of a different material from said metal alloy; and
k. coating said coating material on at least a portion of an outer surface of one or more of said medical devices.

2. The method as defined in claim 1, wherein one or more portions of said medical device body is formed of metal that includes at least one of a) stainless steel, b) CoCr alloy, c) TiAlV alloy, d) aluminum alloy, e) nickel alloy, f) titanium alloy, g) tungsten alloy, h) molybdenum alloy, i) copper alloy, j) beryllium-copper alloy, k) titanium-nickel alloy, l) refractory metal alloy, or m) metal alloy that is modified to further include at least 5 atomic weight percent (awt. %) or atomic percent (awt. %) rhenium and/or hafnium.

3. The method as defined in claim 1, wherein said enhancement material is formulated to i) provide nitric oxide, and/or ii) promote generation of nitric oxide; said enhancement material is at least partially formulated of oxynitride.

4. The method as defined in claim 1, wherein said medical device includes no more than 0.1 wt. % nickel, no more than 0.1 wt. % chromium, and/or no more than 0.1 wt. % cobalt.

5. A method and process for manufacturing medical devices wherein a portion or all of the formed medical device is formed of a metal alloy that includes at least 5 atomic percent of hafnium and/or rhenium; said method comprises:
a. providing rhenium and/or hafnium powder and one or more metal powder additives selected from the group of molybdenum, calcium, chromium, cobalt, copper, gold, iron, magnesium, nickel, niobium, osmium, platinum, rare earth metals, rhenium, silver, tantalum, technetium, titanium, tungsten, vanadium, yttrium, zinc, and/or zirconium, and/or a powder alloy of such metals; said metal powder particles are less than 1000 microns in size;
b. consolidating said metal powder under pressure to form a green part or component;
c. sintering said green part or component;
d. cutting the metal plate to partially or fully form one or more medical devices from the metal plate; and
e. bending one or more of said medical devices into its final or near final shape.

6. The method as defined in claim 1, wherein said metal alloy used to form said medical devices has a) a maximum ion release of a primary component of said metal alloy when inserted or implanted on or in a body of a patient of no more than 0.5 μg/cm2 per day; and wherein said primary component is a component of said metal alloy that constitutes at least 2 wt. % of said metal alloy; b) said metal alloy has an absolute increase in ion release per dose of said metal alloy in the tissue about the implanted medical device of no more than 50 days after inserted or implanted on or in the body of the patient; c) said metal alloy has no more than 50% of the allowed daily exposure of primary metal from said metal alloy during the first 5 days after inserted or implanted on or in the body of the patient; and/or d) a hydrophilicity wherein a contact angle of a water droplet on a surface of said metal alloy is 25-45°.
